(19) United States
(12) Patent Application Publication
(10) Pub. No.: US 2024/0294588 A1
(43) Pub. Date: Sep. 5, 2024

(54) C2 Domain Therapeutics and Uses Thereof

(71) Applicant: University of Maryland, Baltimore, Baltimore, MD (US)

(72) Inventors: Robert J. Bloch, Baltimore, MD (US); Joaquin Muriel, New Freedom, PA (US); Valeriy Lukyanenko, Lutherville, MD (US); Noah Weisleder, Bexley, OH (US)

(73) Assignee: University of Maryland, Baltimore, Baltimore, MD (US)

(21) Appl. No.: 18/567,959

(22) PCT Filed: Jun. 7, 2022
(86) PCT No.: PCT/US22/32549

§ 371 Date: Dec. 7, 2023

Related U.S. Application Data
(60) Provisional application No. 63/197,550, filed on Jun. 7, 2021.

(51) Int. Cl. C07K 14/47; A61K 38/00; C12N 9/12; C12N 15/86

(52) U.S. Cl. CPC C07K 14/4707; C12N 9/12; C12N 15/86; A61K 38/00; C07K 2319/01; C12Y 207/11013

(57) ABSTRACT

Provided herein are fusion proteins engineered from a dysferlin C2 domain sequence linked to a sequence of a homologous fusion partner, vector constructs with cDNA encoding the fusion proteins and viral vectors with the vector constructs and a promoter to control expression thereof. Also provided are methods for treating a dysferlinopathy in a subject in need thereof, for suppressing pathogenic Ca²⁺ signaling in a dysferlinopathic muscle and for targeting proteins to triad junctions in skeletal muscles all utilizing at least the fusion proteins.

### CROSS-REFERENCE TO RELATED APPLICATIONS

This international application claims benefit of priority under 35 U.S.C. § 119(e) of provisional application U.S. Ser. No. 63/197,550, filed Jun. 7, 2021, the entirety of which is hereby incorporated by reference.

### BACKGROUND OF THE INVENTION

### Field of the Invention

The present invention relates to the fields of viral vector and fusion protein construction and muscle pathophysiological conditions. More particularly, the present invention relates viral vectors, viral constructs and fusion proteins effective as therapeutics against dysferlinopathies.

### Description of the Related Art

Dysferlin is a ˜230 kDa protein that is mutated or missing in several muscular dystrophies, including Limb Girdle Muscular Dystrophy type 2B (LGMD2B) and Miyoshi Myopathy (MMD1). Like many large proteins, dysferlin (DYSF) is modular in structure, composed of 7 C2 domains flanking several Fer and DysF domains that extend into the cytoplasm. These are anchored to the membrane of the transverse tubules (TT) at or very near triad junctions (TJs) by a 23 amino acid transmembrane (TM) sequence, followed by a short C-terminal sequence, which extends into the lumen of the transverse tubules.

Nearly all dysferlinopathy patients are compound heterozygotes and show mutations along the entire length of the ORF, resulting in premature stop codons, missense mutations or defective splicing. Many of these point mutations result in pathological changes in skeletal muscle, as assayed in two distinct ways: diminished repair of the sarcolemmal membrane after injury, and faulty stabilization of Ca²⁺ signaling, to produce Ca²⁺ waves. Ca²⁺ waves are indicative of Ca²⁺-induced Ca²⁺ release (CICR), which is associated with skeletal myopathies.

Pathogenic mutations in the DYSF gene occur in nearly all regions of the open reading frame, in sequences encoding all of DYSF's structural domains (FIG. 1), as well as in intronic regions. The moderate homology of the most N-terminal C2 domain, C2A, to one of the C2 domains of synaptotagmin (SYT-1) suggested that dysferlin may play a role in membrane fusion events associated with the repair of damaged sarcolemma. Evidence for this is supported in vitro studies of dye uptake in muscle fibers wounded by laser illumination.

A role for dysferlin in sarcolemmal repair was congruent with its immunolocalization to the sarcolemma in unfixed muscle samples, its increased concentration at sarcolemmal wounds, and the fact that membrane vesicles accumulate in the cortical cytoplasm in dysferlin-null human and mouse muscle (6; 50). Subsequent studies confirmed an association of dysferlin with other repair proteins, including annexins, as well as caveolin 3 (Cav3) and TRIM72/MG53. These results suggest that pathogenesis in dysferlin-null muscle is caused at least in part by defective membrane repair.

Recent studies suggested an alternative role at the triad junction, which was studied after it was found that dysferlin is not necessary for membrane repair in skeletal muscle subjected to eccentric injury. A role for dysferlin at the triad junction was congruent with the observation that dysferlin concentrates in the transverse tubule membranes of perfusion-fixed muscle and of living muscle fibers, rather than at the sarcolemma. There, it colocalizes with the ryanodine receptors (RyR1) in the terminal cisternae of the SR, suggesting that it is a component of triad junctions (TJs). Furthermore, voltage-induced Ca²⁺ transients are lower in amplitude in DYSF-null fibers compared to WT. When they are subjected to mild hypo-osmotic shock injury (OSI) in vitro, dysferlin-null fibers show poor recovery of the Ca²⁺ transient, and those transients that do appear often appear as Ca²⁺ waves, indicative of CICR. Restoration of wild type dysferlin restores the amplitude of Ca²⁺ transients to WT levels and protects against the loss of amplitude and the appearance of waves after OSI. Reagents that block L-type Ca²⁺ channels (LTCC, DHPR) and RyR1, such as diltiazem and dantrolene, also prevented these changes. These results are consistent with the idea that dysferlin plays a key role at TJs in stabilizing DHPR-RyR1 coupling that is essential for Ca²⁺ release in healthy skeletal muscle, and that its absence may result in CICR, which can be pathogenic to skeletal muscle.

Defects in the stabilization of Ca²⁺ in Dysf-null muscle may underlie the defects in membrane repair. The approach entailed measuring the changes in Ca²⁺ signaling and membrane repair in variants of DYSF that lacked individual C2 domains. The results suggested that the two processes were largely co-dependent, as deletion of most of the C2 domains affected both membrane repair and Ca²⁺ signaling. In particular, deletion of C2A (DYSF-AC2A) completely prevented the recovery of the Ca²⁺ transient after OSI, while completely inhibiting membrane repair and the generation of Ca²⁺ waves.

Dysferlinopathies (LGMD2B, MMD1 and other, rarer presentations) affect approximately ˜1 in 100,000 individuals worldwide and are the third most studied form of limb girdle type 2 dystrophy, after LGMD2A and LGMD2K. They are among the best understood autosomal recessive diseases of muscle. Determining their underlying pathology may therefore provide important insights into many forms of muscular dystrophy. Likewise, therapeutics for the dysferlinopathies may prove applicable to muscle disease in general.

There is, therefore, a need in the art for treatments for muscle diseases, for example, dysferlinopathies. Specifically there is a need for therapeutic compositions that restore activity in membrane repair and Ca²⁺ signaling in myofibers affected by muscle diseases. The present invention fulfills this long-standing need and desire in the art.

### SUMMARY OF THE INVENTION

The present invention is directed to a fusion protein engineered from a dysferlin C2 domain sequence linked to a sequence of a homologous fusion partner.

The present invention also is directed to a vector construct comprising a cDNA encoding the fusion protein as described herein.

The present invention is directed further to a viral vector comprising the vector construct as described herein and a promoter effective to control expression of the fusion protein therein.

The present invention is directed further still to a method for treating a dysferlinopathy in a subject in need thereof. In the method a therapeutic amount of a viral vector that encodes a fusion protein comprising a dysferlin C2 domain sequence linked to a sequence of a homologous fusion partner is administered at least once to correct defects in a dysferlinopathic muscle, thereby treating the dysferlinopathy.

The present invention is directed further still to a method for suppressing pathogenic Ca²⁺ signaling in a dysferlinopathic muscle. In the method a fusion protein of a dysferlin C2 domain linked to a homologous fusion partner effective to target at least one triad junction in a dysferlinopathic muscle is delivered thereto. The dysferlin C2 domain sequence is activated upon targeting to at least one triad junction to regulate Ca²⁺ signaling.

The present invention is directed further still to a method for suppressing pathogenic defects during membrane repair in a dysferlinopathic muscle. In the method the dysferlinopathic muscle is contacted with a fusion protein of a dysferlin C2 domain linked to at least one homologous C2 domain. The dysferlinopathic muscle is transfected with a viral vector encoding the fusion protein to express the same.

The present invention is directed further still to a method for targeting proteins to triad junctions in skeletal muscles. In the method a viral vector is engineered that encodes from a single cDNA encoding a fusion protein comprising a protein sequence of interest linked to a sequence homologous to the protein sequence that specifically targets the triad junctions. The viral vector is delivered to the skeletal muscles and the fusion protein is encoded from the single cDNA, where the fusion protein is targeted to the triad junctions via the sequence homologous to the protein sequence.

Other and further aspects, features, benefits, and advantages of the present invention will be apparent from the following description of the presently preferred embodiments of the invention given for the purpose of disclosure.

### BRIEF DESCRIPTION OF THE FIGURES

The appended drawings have been included herein so that the above-recited features, advantages and objects of the invention will become clear and can be understood in detail. These drawings form a part of the specification. It is to be noted, however, that the appended drawings illustrate preferred embodiments of the invention and should not be considered to limit the scope of the invention.

FIG. 1 is a schematic of the structure of DYSF.

FIGS. 2A-2E show the DYSF-C2A distribution and partial protection against loss of Ca²⁺ transient and development of Ca²⁺ waves after OSI. The construct in FIG. 2A was electroporated into Flexor digitorum brevis (FDB) muscles of dysferlin-null A/J mice and imaged 10 d later. FIG. 2B shows accumulation primarily at Z-disks (3 are indicated with arrows). FIG. 2C shows Ca²⁺ transients registered with Rhod-2 in a myofiber expressing DYSF-C2A before and 5 min after osmotic shock injury. Distribution of the Venus construct is indicated to the right. Recovery is partial (in the upper region of fiber). FIG. 2D quantitates results from several dozen fibers of each type. Fibers expressing DYSF-C2A are intermediate between Venus (negative control) and WT DYSF (positive control) controls but show a low frequency of Ca²⁺ waves, only slightly more than with WT DYSF. FIG. 2E shows the recovery of the transient (as normalized Ca release) and Ca wave frequency as a function of the amount of Ven-DYSF-C2A present in each fiber. Higher levels of expression show more consistent recoveries and suppression of waves than lower levels.

FIGS. 3A-3B show the membrane repair by the DYSF-C2A domain. FDB muscles of A/J mice were electroporated with expression plasmids for WT DYSF or DYSF-C2A. 7d later muscles were removed and injured by infrared laser illumination in the presence of FM4-64 lipophilic dye. FIG. 3A shows FM4-64 fluorescence at the injury site as f(t). FIG. 3B shows areas under the curves from the data in A show that the DYSF-C2A improved repair as well as WT DYSF. Means±SEM, n=7, *p<0.01 by ANOVA followed with Tukey's post-hoc test.

FIGS. 4A-4E show that C2-PKCα concentrates at TJs and as a fusion with DYSF-C2A promotes Ca²⁺ signaling. The constructs in FIG. 4A were electroporated and visualized as in FIG. 2. FIG. 4B shows that the C2 domain of PKCα concentrates at the level of A-I junctions, probably at TJs (B1, arrows) and drives DYSF-C2A to accumulate there (B2, arrows). FIG. 4C shows Ca transients visualized with Rhod-2 in a fiber expressing the construct in A2. Recovery is complete. FIG. 4D quantitates results from several dozen fibers transfected with each construct. C2-PKCα-DYSF-C2A gives results identical to WT DYSF. FIG. 4E shows the recovery of the transient (normalized Ca release) and Ca wave frequency as a function of the amount of Ven-C2-PKCα-DYSF-C2A in each fiber. Note the high level of Ca release and low frequency of waves indicated by the y-intercepts at low transfection levels.

FIGS. 5A-5B show membrane repair by C2-PKCα-DYSF-C2A as in FIGS. 3A-3B. FIG. 5A shows FM4-64 fluorescence at the injury site as f(t). FIG. 5B shows areas under curves from FIG. 5A show that the C2-PKCα-DYSF-C2A improved repair as well as WT DYSF. Means±SEM, n=7, *p<0.01 by ANOVA followed with Tukey's post-hoc test.

FIG. 6 shows the distribution of C2 domain constructs in Z-disks vs TJs.

FIGS. 7A-7B show that Dysferlin codistributes with PKCα at or near TJs in skeletal myofibers (FIG. A) and co-IPs with anti-PKCα from muscle extracts and HEK293 cells that express both proteins (FIG. 7B). Control IgG used in co-IP did not yield PKCα bands.

FIG. 8 shows the effect of PMA and staurosporine on Ca²⁺ transients in A/J fibers, measured as in FIGS. 2A-2E.

FIGS. 9A-9C show the effect of BAPTA-AM on Ca²⁺ transients in control A/JCr fibers and in A/J fibers before (FIG. 9A) and after (FIG. 9B) OSI, and on Ca²⁺ wave frequency after OSI (C), as measured as in FIG. 2. *P<0.05 compared to Con. **P<0.05 compared to A/J. (ANOVA)

FIGS. 10A-10D show that GCaMP6fu-DYSF-ΔC2A concentrates at TJs and stabilizes Ca²⁺ signaling. The construct in FIG. 10A was electroporated and visualized. FIG. 10B shows the chimeric construct concentrates at the level of A-I junctions, probably TJs (arrows). FIG. 10C shows Ca²⁺ transients visualized with Rhod-2 in a myofiber expressing the construct in FIG. 10A. Recovery is complete. FIG. 10D quantitates results from several dozen fibers transfected with each construct indicated. Fibers expressing Ven-GCaMP6fu-DYSF-ΔC2A are identical to those expressing Ven-WT DYSF by ANOVA. (They do not show frequent Ca²⁺ waves). The results suggest that placing a Ca²⁺-binding moiety at TJs protects against the loss of the transient after OSI.

FIGS. 11A-11C show GCaMP6fu linked to the N-terminus of DYSF (FIGS. 11A-11B) or DYSF-ΔC2A (FIG. 11C) senses changes in local [Ca²⁺ ] and, in FIG. 11C, protects against the loss of the Ca²⁺ transient after OSI.

FIGS. 12A-12B shows the localization of C2 PKCα in control C57Bl/6 fibers (FIG. 12A) and A/J fibers (FIG. 12B) via images of a Venus-tagged version of the C2 domain of PKCα, Venus-C2-PKCα.

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method described herein can be implemented with respect to any other method described herein.

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, “comprise” and its variations, such as “comprises” and “comprising,” will be understood to imply the inclusion of a stated item, element or step or group of items, elements or steps but not the exclusion of any other item, element or step or group of items, elements or steps unless the context requires otherwise. Similarly, “another” or “other” may mean at least a second or more of the same or different claim element or components thereof.

In one embodiment of the present invention there is provided a fusion protein engineered from a dysferlin C2 domain sequence linked to a sequence of a homologous fusion partner.

In this embodiment the dysferlin C2 domain sequence may be an N-terminal sequence or a C-terminal sequence. In one aspect of this embodiment the dysferlin C2 domain sequence may be an N-terminal C2A domain sequence (Dysf-C2A). In another aspect of this embodiment the homologous fusion partner may comprise a sequence from at least one C2 domain of an α isoform of protein kinase C (C2-PKCα). In this embodiment and both aspects thereof the fusion protein may be an engineered C2-PKCα-DYSF-C2A fusion protein.

In another embodiment of the present invention there is provided a vector construct comprising a cDNA encoding the fusion protein as described supra.

In yet another embodiment of the present invention there is provided a viral vector comprising the vector construct of claim 5 and a promoter effective to control expression of the fusion protein therein. In this embodiment the promoter may be a muscle-specific promoter.

In yet another embodiment of the present invention there is provided a method for treating a dysferlinopathy in a subject in need thereof, comprising administering to the subject at least once a therapeutic amount of a viral vector that encodes a fusion protein comprising a dysferlin C2 domain sequence linked to a sequence of a homologous fusion partner to correct defects in a dysferlinopathic muscle, thereby treating the dysferlinopathy.

In this embodiment the homologous fusion partner may target the dysferlin C2 domain sequence to triad junctions in a skeletal muscle. Also in this embodiment the fusion protein may comprise the C2A domain of dysferlin and at least one C2 domain of an α isoform of protein kinase C (C2-PKCα-DYSF-C2A). In addition the dysferlinopathy may be muscular dystrophy.

In yet another embodiment of the present invention there is provided a method for suppressing pathogenic Ca²⁺ signaling in a dysferlinopathic muscle, comprising delivering a fusion protein of a dysferlin C2 domain linked to a homologous C2 domain effective to target at least one triad junction in a dysferlinopathic muscle; and activating the dysferlin C2 domain sequence upon targeting to the at least one triad junction to regulate Ca²⁺ signaling.

In this embodiment the delivering step may comprise contacting the dysferlinopathic muscle with a viral vector encoding the fusion protein. Also in this embodiment the pathogenic Ca²⁺ signaling may occur in muscular dystrophy. In addition the fusion protein comprises the C2A domain of dysferlin and at least one C2 domain of an α isoform of protein kinase C (C2-PKCα-DYSF-C2A). Particularly, the fusion protein is a C2-PKCα-DYSF-C2A fusion protein.

In yet another embodiment of the present invention there is provided a method for suppressing pathogenic defects during membrane repair in a dysferlinopathic muscle, comprising contacting the dysferlinopathic muscle with a fusion protein of a dysferlin C2 domain linked to at least one homologous C2 domain.

In this embodiment the contacting step may comprise transfecting the dysferlinopathic muscle with a viral vector encoding the fusion protein to express the same. Also in this embodiment the fusion protein may comprise the C2A domain of dysferlin and at least one C2 domain of an α isoform of protein kinase C (C2-PKCα-DYSF-C2A). In addition the dysferlinopathic muscle may be a muscle affected by muscular dystrophy.

In yet another embodiment of the present invention there is provided a method for targeting proteins to triad junctions in skeletal muscles, comprising engineering a viral vector that encodes from a single cDNA encoding a fusion protein comprising a protein sequence of interest linked to a sequence homologous to the protein sequence that specifically targets the triad junctions; delivering the viral vector to the skeletal muscles; and encoding the fusion protein from the single cDNA, said fusion protein targeted to the triad junctions via the sequence homologous to the protein sequence.

In this embodiment the encoding step may be under the control of a muscle-specific promoter in the viral vector. Also in this embodiment the fusion protein may comprise the C2A domain of dysferlin and at least one C2 domain of an α isoform of protein kinase C (C2-PKCα-DYSF-C2A).

The present invention demonstrates that DYSF-C2A is unique and when targeted to the triad junction via a novel, engineered fusion partner, it can correct the defects in Ca²⁺ signaling and sarcolemmal membrane repair typical of dysferlinopathic muscle. Dysferlin is missing or mutated in several forms of muscular dystrophy (e.g., LGMD2B, MMDI). The absence of dysferlin or the presence of dysferlin mutants linked to myopathology is associated with changes in calcium signaling.

By exploring different variants, the most N terminal C2 domain of dysferlin is essential to maintain normal calcium signaling, and that overexpression of that domain, termed “C2A”, suppresses pathogenic calcium signaling in dysferlin-null muscle fibers. The C2 domain of protein kinase Ca can promote the association of C2A or tandem C2AC2A constructs to the triad junction, where dysferlin normally functions. The chimaeric protein, consisting of pieces of PKCα and dysferlin's C2A domains are the most effective reagents found to suppress pathogenic calcium signaling and in restoring normal membrane repair to dysferlin-null myofibers. Compromised calcium signaling and membrane repair are not only associated with dysferlinopathies but also linked to many different forms of muscular dystrophy. The chimaeric PKCα/C2AC2A construct of the present invention is useful in suppressing the pathology of dysferlinopathic muscle fibers in vitro.

The present invention teaches that engineered fragments of the DYSF protein can be designed to target the TJs and correct the defects in membrane repair and Ca²⁺ signaling associated with disease. In vitro, reintroduction of WT dysferlin into dysferlin-null myofibers restores both normal membrane repair and normal Ca²⁺ signaling. Dysferlin's C2A domain plays a unique role and is essential for both activities. The C2A domain of dysferlin is remarkable in that it bears significant homology to only a small number of C2 domains of other proteins, but not to the other C2 domains of dysferlin. By contrast, the C2E domain of dysferlin is much more homologous to sequences in myoferlin, as well as in Fer-1.

Upon examining the C2A domain on its own, although distributed widely in the myoplasm, it could support membrane repair and Ca²⁺ signaling to almost normal levels. The potency of the C2A domain in these assays increased when it was targeted to the triad junctions of myofibers by linking it to one of the few structures with which it shares homology, the C2 domain of the a isoform of protein kinase C (C2-PKCα). Chimeric constructs of the C2A of dysferlin (Dysf-C2A) and C2-PKCα restore complete activity in membrane repair and Ca²⁺ signaling in dysferlin-null myofibers in vitro, and they do so efficiently, even when expressed at relatively low levels. The present invention shows that the C2A domain of dysferlin, targeted to TJs, is a potent, efficient and stable replacement for WT dysferlin in dysferlinopathic muscle.

The present invention shows:
(i) optimizing the efficacy and specificity of different engineered constructs of DYSF-C2A in rescuing the deficits in Ca²⁺ signaling and sarcolemmal membrane repair seen in Dysf-null muscle in vitro;
(ii) identifying the mechanism of targeting of the chimeric constructs to the triad junction; and
(iii) testing if Ca²⁺ binding by these constructs at the triad junction is sufficient to account for their activity.

Dysferlinopathies remain one of >50 muscular dystrophies without a treatment or a cure. With an ORF of 6.3 kb, DYSF is too large to package in AAV, a common vector used for gene therapy of muscle diseases. “Nanodysferlins”, i.e., variants of dysferlin missing several of its C2 domains, are at least partially active, but they neither target TT nor support normal Ca²⁺ signaling. The methods of the present invention avoid difficulties in AAV packaging by using ORFs less than <2.5 kb and improve transduction efficiency, opening a new avenue for possible treatment of dysferlinopathies. Inadequate membrane repair and the destabilization of the DHPR-RyR1 complex, increasing Ca²⁺ leak and the frequency of CICR, which are all common to other diseases of muscle allows the present invention to be applicable to other forms of muscular dystrophy.

The methods of the present invention use a cDNA with an ORF<2.5 kb, requires only one AAV construct, does not require recombination in situ, yields efficient expression of transgenes which effectively protect against the two well-known defects of dysferlin-null muscle, susceptibility to membrane damage due to faulty membrane repair, and destabilization of the Ca²⁺ transient and the appearance of Ca²⁺ waves.

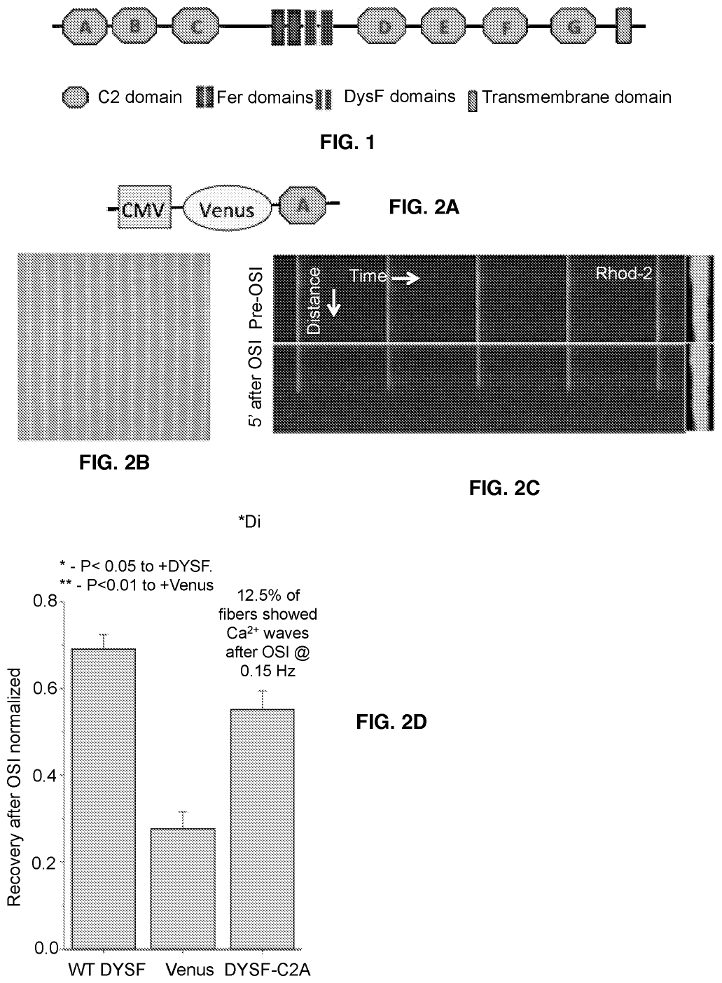

The methods of the present invention utilizes the unique features of dysferlin's C2A domain, the most N-terminal C2 domain, which has limited homology to other C2 domains (FIG. 1, Table 1; a type 2 C2 domain).

TABLE 1
Uniqueness of DYSF C2A Domain

| | Similarity (%)* | | |
|---|---|---|---|
| Protein C2 domain (man) [symbol] | Id | Hom | Tot |
| DYSF-C2A | 100 | 0 | 100 |
| DYSF-C2B | 31 | 13 | 44 |
| DYSF-C2C | 25 | 18 | 43 |
| DYSF-C2D | 26 | 13 | 39 |
| DYSF-C2E | 29 | 14 | 43 |
| DYSF-C2F | 29 | 20 | 49 |
| DYSF-C2G | 22 | 13 | 35 |
| Myoferlin** C2A | 43 | 18 | 61 |
| Fer1-like** 3 C2A | 43 | 18 | 61 |
| Otoferlin** C2A | 25 | 23 | 48 |
| PKCα | 35 | 23 | 58 |
| Rabphilin-3A X2 | 30 | 22 | 52 |
| SYT-1 | 31 | 16 | 47 |

[symbol]

domain boundaries are approximate;
*id, identical; Hom, homologous; Tot, total;
**ferlin family member

When expressed on its own in dysferlin-null A/J myofibers (as a Venus fusion protein), the C2A domain can protect against the loss of membrane repair and the destabilization of the Ca²⁺ transient, but it is more active if it is targeted more efficiently to the triad junctions (TJs) with another C2 domain, that of PKCα. Increased targeting to the TJs allows the engineered constructs of the present invention to be fully active at lower intracellular concentrations, which minimizes the amounts of virus needed for therapy thus reducing the immune response to AAV and viral toxicity.

The methods of the present invention target the C2A domain of dysferlin to the TJs by placing it in tandem with the C2 domain of PKCα. The PKCα-C2 domain is inactive in assays of membrane repair and Ca²⁺ signaling, however, suggesting that its contribution to the results may be limited to its ability to concentrate at TJs. PKCα also concentrates at or near the TJs of skeletal muscle, where it binds to dysferlin.

The present invention indicates that full length dysferlin and its variants are only active when they are concentrated at or very near the TJs, but that this alone is probably not sufficient for full activity. In particular, its ability to bind Ca²⁺ at its N-terminal region, via C2A, may also be necessary. This idea has been strengthened by the finding that replacing the C2A domain with GCaMP6fu yields a dysferlin variant that concentrates at TJs and that has full activity in assays of Ca²⁺ signaling. These results elucidate the possible roles of the small but significant increases in junctional (as well as myoplasmic) Ca²⁺ in dysferlinopathies and in other diseases of muscle. These transgene constructs, expressed in AAV, are effective in countering the effects of dysferlinopathy, and also may be useful in suppressing the abnormal regulation of Ca²⁺ in other forms of muscular dystrophy.

The methods and constructs of the present invention have several innovative features.
(i) It illustrates the relationship between the two functions of dysferlin, membrane repair and stabilization of Ca²⁺ signaling to assess their possible interdependence.
(ii) The engineered constructs of the present invention have the unique ability to replicate the membrane repair and Ca²⁺ signaling activities of intact dysferlin. They are small enough (30-50 kDa in mass) to be easily expressed via AAV transduction.
(iii) The constructs of the present invention are efficient at low intracellular concentrations because they are targeted to TJs via a novel fusion partner. High efficiency allows lower viral doses required for therapy.
(iv) The methods of the present invention indicate the existence of a novel mode of targeting proteins to TJs.

### Example 1

### Results

### Activity of the C2A Domain

It is known that both membrane repair and stabilization of Ca²⁺ signaling are compromised in dysferlin-null A/J muscle fibers, and that the introduction of WT dysferlin by electroporation restores full activity but only in the region of the fibers in which dysferlin is expressed. Furthermore, mutant dysferlins, missing individual C2 domains or carrying pathogenic point mutations fail to restore full activity, although some of these mutants accumulate normally at TJs. The mutant used here is DYSF-ΔC2A, i.e., DYSF missing its C2A domain. This mutant concentrates in TT at TJs but is inactive in both membrane repair and Ca²⁺ signaling. C2A has novel activities that include binding to Ca²⁺, lipids and other proteins, including TRIM72/MG53.

Whether replacing the C2A domain of dysferlin (AF075575.1, amino acids 1-100) with other, partially homologous C2 domains would restore activity was first examined, but neither the C2A domain of myoferlin (61% homologous) nor the C2 domain of PKCα (58% homologous) were effective. Congruent with these results, two pathogenic mutations in C2A, V67D and W52R, inactivated full length dysferlin, whereas 2 polymorphisms, V68L and A84V, left dysferlin's Ca²⁺ signaling activity intact.

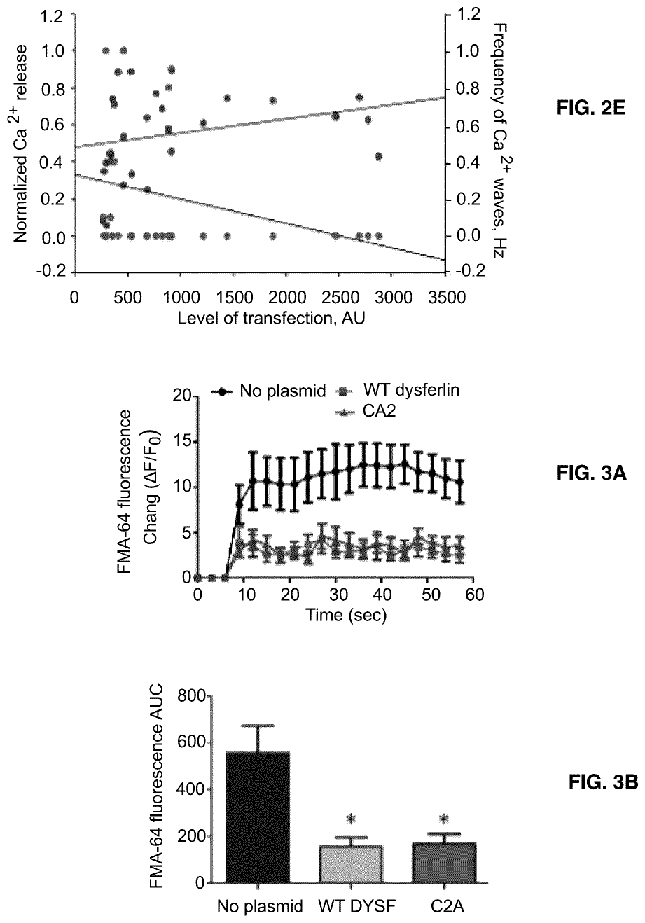

Based on these results, the C2A domain of dysferlin (DYSF-C2A) was examined on its own, expressed as a Venus fusion protein (FIG. 2A) partially restored some Ca²⁺ signaling activity (FIGS. 2C-2E), though, unlike WT dysferlin, it distributed throughout the myoplasm and did not concentrate at TJs (FIG. 2B). Based on the fluorescence intensity of Venus, it was very well expressed, with maximum intensities approaching 3000 AU (Arbitrary Units) and mean intensities of ˜1100, comparable to those reached by WT dysferlin (FIG. 2E). (Both constructs were compared after transfection with 1.2 μg/ml plasmid DNA.) DYSF-C2A also fully stabilized membrane repair activity (FIGS. 3A-3B). Like WT dysferlin, introduction of pathogenic mutations into the isolated C2A domain inhibited its activity in Ca²⁺ signaling, and other isolated C2 domains (MYOF-C2A, PKCα-C2, DYSF-C2B, DYSF-C2C, DYSF-C2E) failed to replicate the effects of DYSF-C2A. These results strongly suggest that DYSF-C2A, expressed on its own at high levels in DYSF-null muscle fibers, can replace WT dysferlin and that its effect is specific.

### Activity of the C2 Domain of PKCα in Ca²⁺ Signaling

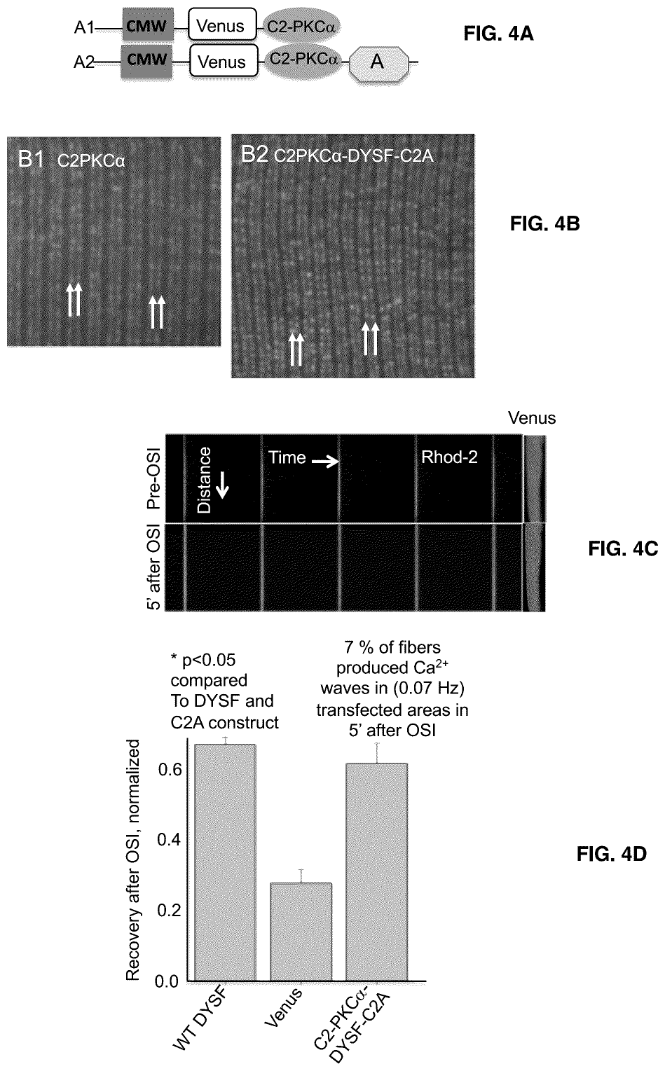

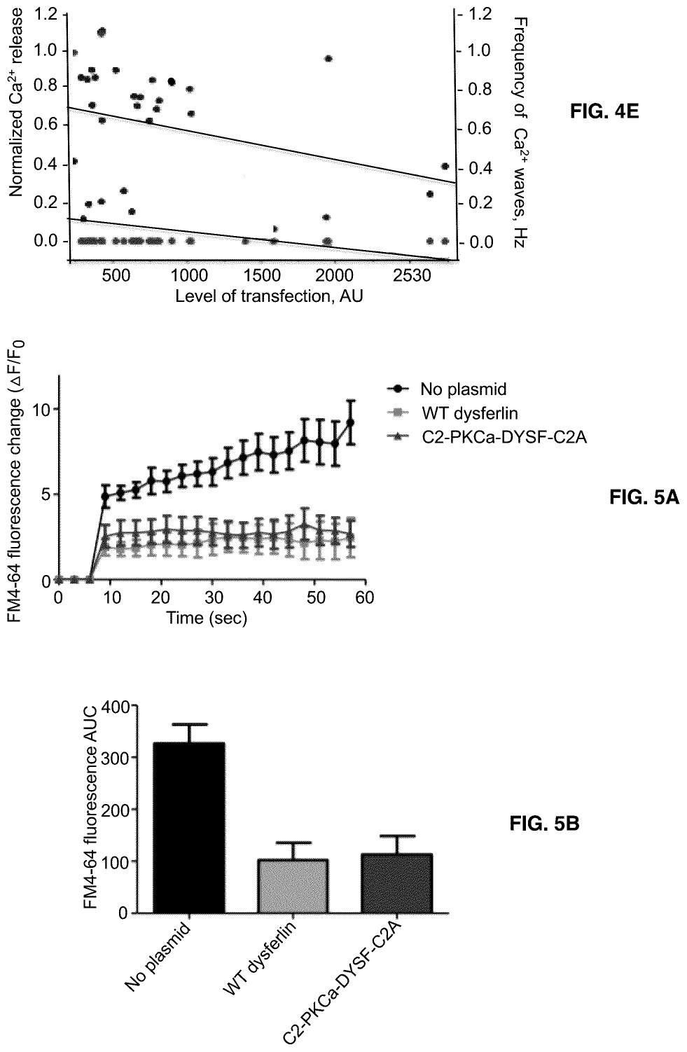

Although it was completely without activity in the Ca²⁺ signaling assays, the C2 domain of PKCα (XP_024306597.1, residues 71-229), tagged with a Venus moiety (FIG. 4A1) concentrated at the level of the A-I junctions of A/J myofibers (FIG. 4B1), like WT DYSF. None of the other isolated C2 domains assayed did this, suggesting that the PKCα-C2 domain recognizes other proteins present at TJs and that it might serve as a vehicle for more efficiently targeting the C2A domain of dysferlin to the TJs.

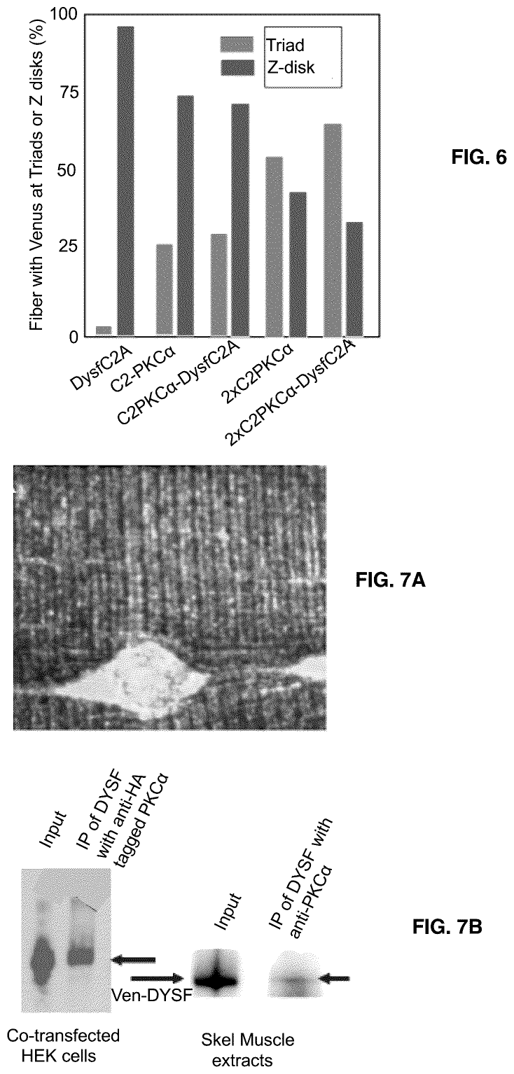

A chimeric construct was created with C2-PKCα just N-terminal to DYSF-C2A (tagged with Venus: FIG. 4A2). This construct targeted the TJ regions in ˜25% of transfected fibers FIG. 4B2; FIG. 6), with the remaining fibers showing primarily Z-disk localization, as with DYSF-C2A alone. The C2-PKCα-DYSF-C2A construct's ability to stabilize Ca²⁺ signaling (FIG. 4C), was indistinguishable from WT DYSF in restoring the amplitude of the Ca²⁺ transient and almost as effective in suppressing Ca²⁺ waves (FIGS. 4D-4E). Notably, it was more effective than C2A alone at lower levels of expression (compare the intercepts of the 2 lines on the y-axes in FIG. 2E, y=0.47 for Ca transient, y=0.37 for wave frequency, and FIG. 4E, y=3.7 for Ca transient, y=0.11 for wave frequency) consistent with the idea that concentrating the DYSF-C2A domain at TJs with C2-PKCα created a more potent construct. As shown in FIGS. 5A-5B, this construct was just as effective as WT DYSF in supporting membrane repair as well.

Increasing targeting of the DYSF-C2A domain to TJs with more potent chimeric C2-PKCα constructs will further increase their activity and allow them to support normal Ca²⁺ signaling and membrane repair at even lower levels of expression. The present invention shows that a construct that contains 2 C2-PKCα domains with a single DYSF-C2A domain confirms that the addition of a second PKCα C2 domain increases the relative number of myofibers with chimeric proteins concentrated at TJs to 75% (FIG. 6).

Association of PKCα with Dysferlin

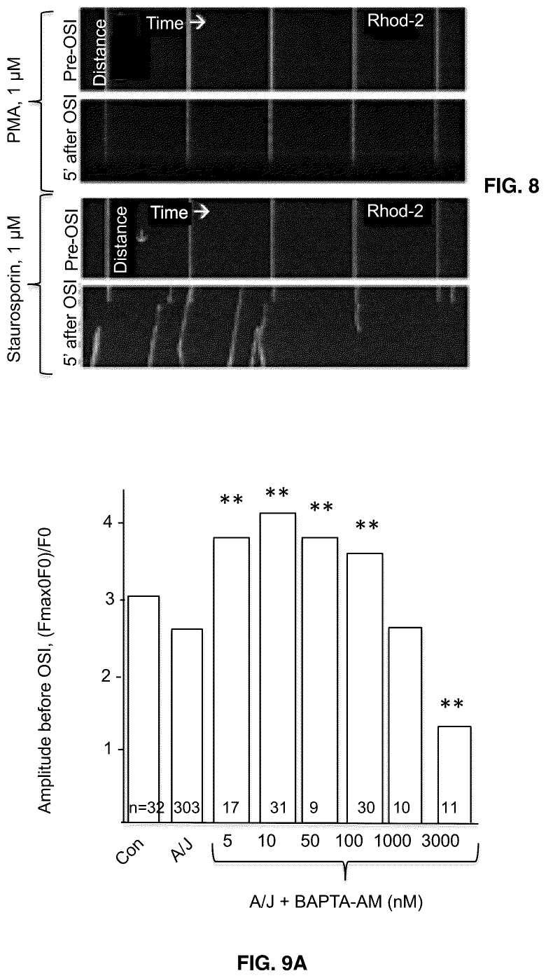

The ability of the C2 domain of PKCα to target dysferlin's C2A domain to TJs raised the possibility that these two proteins might associate in skeletal muscle fibers. Immunofluorescence and co-immunoprecipitation studies shown in FIGS. 7A-7B that the two proteins each concentrate at or near the TJs of skeletal myofibers and they co-immunoprecipitate (co-IP) both from skeletal muscle extracts and from extracts of HEK293 cells that were cotransfected to express both proteins. This is not the case with two other PKC isoforms expressed in skeletal muscle, PKCβ2 and PKCγ, despite the fact that their C2 domains share considerable homology with that of PKCα. The association of PKCα with DYSF is mediated by the latter's central C2 domains (C2C, C2D and C2E). Notably, however, neither phorbol 12-myristate 13-acetate (PMA) nor staurosporine, drugs that activate and inhibit PKCα, respectively, have any effect on the colocalization of dysferlin and PKCα in myofibers or on their ability to co-IP from HEK cells. By contrast, these drugs significantly alter the amplitude of the Ca²⁺ transients (FIG. 8), suggesting that PKCα regulates Ca²⁺ release from the SR in response to voltage, perhaps via its association with dysferlin.

### Elevated Ca²⁺ Levels at TJs Underlies Defects in Ca Signaling in Dysferlinopathy

Studies of many forms of muscular dystrophy have suggested that they share a common feature: elevated levels of Ca²⁺ in the myoplasm. As dysregulation of the Ca²⁺ transient occurs in dysferlin-null A/J myofibers, the possibility was tested that chelating myoplasmic Ca²⁺ with a cell-permeant chelator, BAPTA-AM, would restore the Ca²⁺ transient to control levels in uninjured fibers, where transients are typically reduced in amplitude by ˜15% compared to WT, and would protect the fibers against a loss of the transient and the appearance of Ca²⁺ waves after OSI.

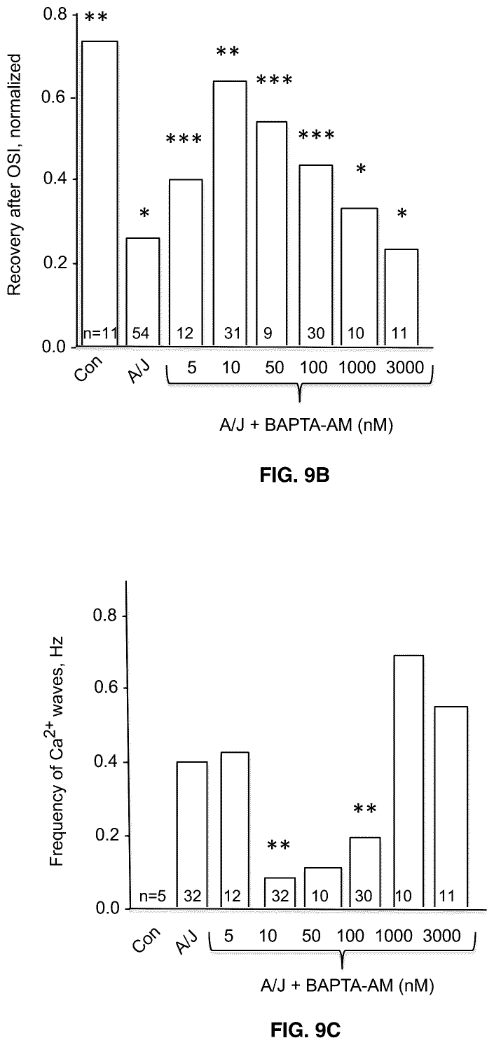

FIGS. 9A-9C show that BAPTA-AM added to cultured myofibers at very low concentrations (10 nM) effectively restores A/J myofibers to the WT phenotype. A fluorescent variant of BAPTA-AM, Fluo4-AM, was used to estimate the amount of the chelator that accumulates in the treated myofibers and it was found that 10 nM extracellular concentrations led to 7-10 fold higher intracellular concentrations under these loading conditions. This concentration of BAPTA in myofibers is sufficient to restore elevated myoplasmic [Ca²⁺ ] of 150-200 nM to levels close to WT levels of ˜100 nM. This can explain why as little as 10 nM BAPTA-AM can restore the WT phenotype.

To assay Ca²⁺ transients, Rhod-2 was added to myofibers as the Rhod2-AM derivative at a concentration of 4.4 μM, or almost 500× higher than the concentration of BAPTA-AM that effectively restores the WT phenotype. Rhod-2 is essentially rhodamine on a BAPTA backbone, so its mode of binding Ca²⁺ is identical to that of BAPTA. Rhod-2 has a (calculated) Stokes' radius ˜20% larger than BAPTA; it has a lower affinity for Ca²⁺ and its solubility in DMSO is ˜20× lower than BAPTA's. Rhod-2 appears to distribute uniformly in the myoplasm under these conditions of loading.

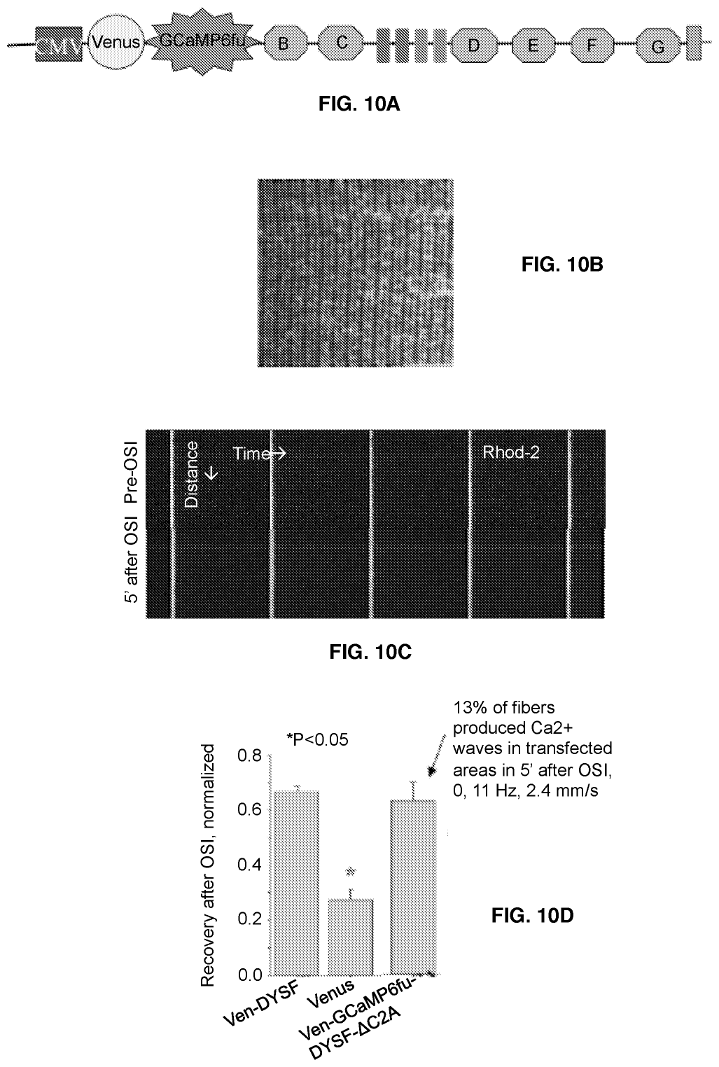

A strategy was used to target a Ca²⁺ chelator directly to the TJ, taking advantage of the unique characteristics of dysferlin's C2A domain. Dysf-AC2A targeted the TJs like the WT protein, but it did not rescue the Ca²⁺ transient after OSI. The substitution of C2A with a high affinity Ca²⁺ binding moiety might not alter TJ targeting but might restore stability to the Ca²⁺ transient and allow one to monitor changes in Ca²⁺ in the junctional cleft. GCaMP6fu, which binds Ca²⁺ rapidly and with high affinity, was used as the Ca²⁺ binding moiety, and placed where C2A is normally found in native dysferlin (FIG. 10A). A Venus tag was used to identify transfected cells (the GCaMP signal is weak unless Ca²⁺ levels increase above background) and the chimeric construct was seen to accumulate at TJs (FIG. 10B). Ca²⁺ transients were measured before and after OSI (FIGS. 10C-10D) and it was found that the presence of the GCaMP6fu moiety in place of dysferlin's C2A domain fully protected the Ca²⁺ transients against loss of amplitude following OSI. By contrast, GCaMP6fu expressed alone in the myoplasm (l,e., not as a dysferlin chimera) was less active in these assays. These results suggest that Ca²⁺ must be elevated specifically at the TJs of A/J myofibers for it to destabilize the Ca²⁺ transient following injury.

### Changes in Ca²⁺ in the TJ

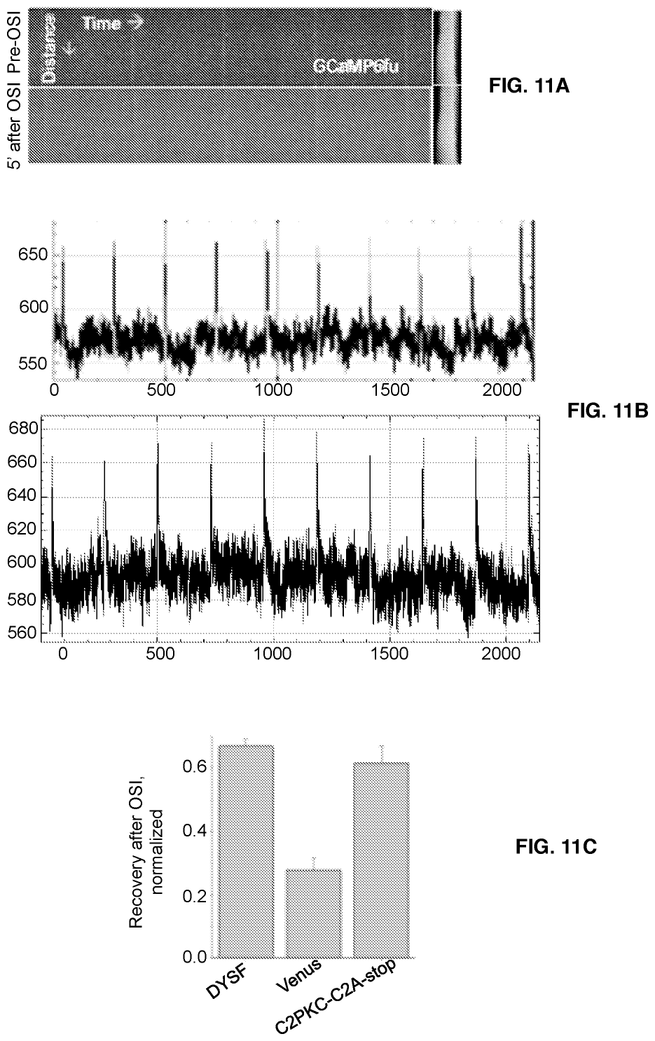

It was next determined if the GCaMP6fu moiety, placed at TJs through linkage to WT DYSF or to DYSF-ΔC2A could detect the changes in Ca²⁺ that occur as the transfected muscle is electrically stimulated. Studies using Venus constructs as well as the DYSF-GCaMP6fu chimeras without Venus showed that the chimeras accumulated like WT DYSF at TJs. FIGS. 11A-11C shows that these chimeras can indeed sense junctional changes in Ca²⁺. In particular, the GCaMP6fu signal in both constructs increases over background whenever the fiber is stimulated (at 1 Hz, as above), and the amplitude of the signal, although low, can be accurately measured. Unlike DYSF-ΔC2A, the chimeric construct prevents the loss of amplitude of the Ca²⁺ transient after OSI, whether measured with Rhod-2 or with the fluorescence changes in DYSF-ΔC2A-GCaMP6fu. As DHPR-RyR1 coupling in otherwise healthy muscle is mechanical, these changes in GCaMP6fu fluorescence should only reflect the local changes in Ca²⁺ at the TJ that occur as the RyR1 channels open. The amount of Ca²⁺ flux thru the RyR1s is not likely to be altered due to the substitution of GCaMP6fu for dysferlin's C2A in the DYSF-ΔC2A-GCaMP6fu chimera, as DYSF-GCaMP6fu yields the same results.

### C2-PKCα Accesses the Triad Junctions of Dysferlin-Null (A/J) Myofibers

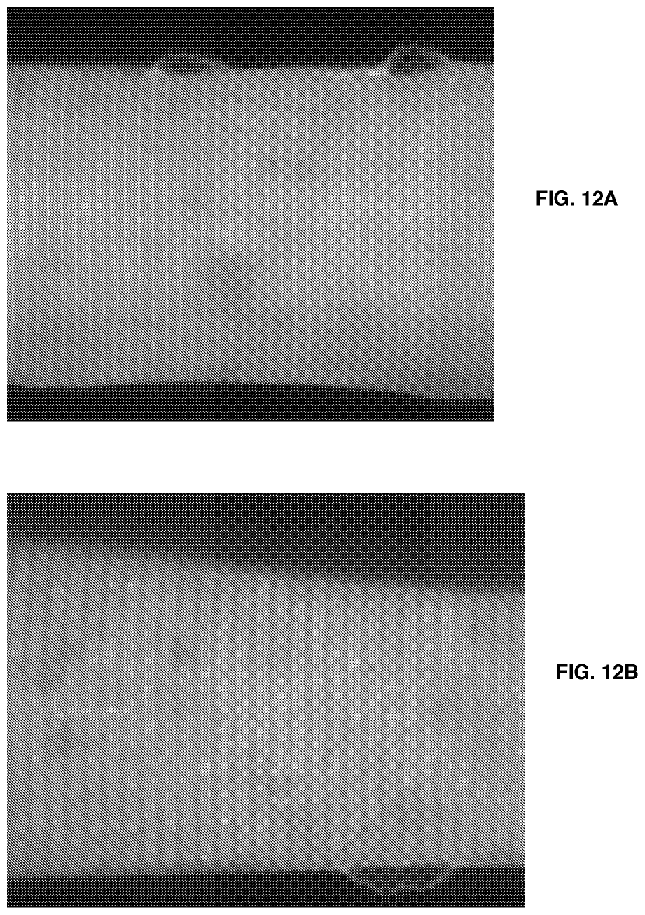

A Venus-tagged version of the C2 domain of PKCα was expressed in both control (C57Bl/6) (FIG. 12A) and dysferlin-null (A/J) myofibers (FIG. 12B) and the transfected fibers were imaged. Ven-C2-PKCα is excluded from the triad junctions of control fibers and concentrates instead at Z-disks (vertical lines). By contrast, it partially accesses the triad junctions of some A/J fibers, where it concentrates at the level of the A-I junction (doublet lines clearly apparent in some places), mostly in puncta, consistent with TJs. A/J fibers that do not show this pattern appear like the controls. The results suggest that the absence of dysferlin causes a change in the triad junction that allows Ven-C2-PKCα to access its very limited volume and to concentrate there. They help to explain the ability of the chimeric constructs containing Ven-C2-PKCα to potentiate the beneficial effects of the DYSF-C2A domain.

### Example 2

### Plasmids

pV-C2Astop Plasmid

Using the primers shown below, Dysferlin C2A domain sequence plus 90 nucleotides downstream of the 3′, were inserted by digestion ligation in the pmVENUS-C1 plasmid (provided by Addgene). The open reading frame includes venus (underlined)-C2A (italics).

### Primers Used for PCR:

DFkpnS (KpnI):
(SEQ ID NO: 1)
CGACggtaccactagtacgcgtATG.
DFdelC2BetcecoVA (EcoRV):
(SEQ ID NO: 2)
ATCAGATATCTCAGCTGAAGGGCTTCACCA
GCACAGCTCCAGGCAGCGGTGTGTAG.
Nucleotide sequence:
SEQ ID NO: 3
ATGGTGAGCAAGGGCGAGGAGCTGTTCACCGGGGTGGTGCCCATCCTGGTCGAGCTG
GACGGCGACGTAAACGGCCACAAGTTCAGCGTGTCCGGCGAGGGCGAGGGCGATGCC
ACCTACGGCAAGCTGACCCTGAAGCTCATCTGCACCACCGGCAAGCTGCCCGTGCCCT
GGCCCACCCTCGTGACCACCCTCGGCTACGGCCTGCAGTGCTTCGCCCGCTACCCCG
ACCACATGAAGCAGCACGACTTCTTCAAGTCCGCCATGCCCGAAGGCTACGTCCAGGA
GCGCACCATCTTCTTCAAGGACGACGGCAACTACAAGACCCGCGCCGAGGTGAAGTTC
GAGGGCGACACCCTGGTGAACCGCATCGAGCTGAAGGGCATCGACTTCAAGGAGGAC
GGCAACATCCTGGGGCACAAGCTGGAGTACAACTACAACAGCCACAACGTCTATATCAC
CGCCGACAAGCAGAAGAACGGCATCAAGGCCAACTTCAAGATCCGCCACAACATCGAG
GACGGCGGCGTGCAGCTCGCCGACCACTACCAGCAGAACACCCCCATCGGCGACGGC
CCCGTGCTGCTGCCCGACAACCACTACCTGAGCTACCAGTCCAAGCTGAGCAAAGACC
CCAACGAGAAGCGCGATCACATGGTCCTGCTGGAGTTCGTGACCGCCGCCGGGATCAC
TCTCGGCATGGACGAGCTGTACAAGTCCGGACTCAGATCTCGAGCTCAAGCTTCGAATT
CTGCAGTCGACggtaccactagtacgcgtATGCTGAGGGTCTTCATCCTCTATGCCGAGAACGT
CCACACACCCGACACCGACATCAGCGATGCCTACTGCTCCGCGGTGTTTGCAGGGGTG
AAGAAGAGAACCAAAGTCATCAAGAACAGCGTGAACCCTGTATGGAATGAGGGATTTGA
ATGGGACCTCAAGGGCATCCCCCTGGACCAGGGCTCTGAGCTTCATGTGGTGGTCAAA
GACCATGAGACGATGGGGAGGAACAGGTTCCTGGGGGAAGCCAAGGTCCCACTCCGA
GAGGTCCTCGCCACCCCTAGTCTGTCCGCCAGCTTCAATGCCCCCCTGCTGGACACCA
AGAAGCAGCCCACAGGGGCCTCGCTGGTCCTGCAGGTGTCCTACACACCGCTGCCTG
GAGCTGTGCTGGTGAAGCCCTTCAGCTGA
Amino acid sequence:
SEQ ID NO: 4
MVSKGEELFTGVVPILVELDGDVNGHKFSVSGEGEGDATYGKLTLKLICTTGKLPVPWPTLV
TTLGYGLQCFARYPDHMKQHDFFKSAMPEGYVQERTIFFKDDGNYKTRAEVKFEGDTLVN
RIELKGIDFKEDGNILGHKLEYNYNSHNVYITADKQKNGIKANFKIRHNIEDGGVQLADHYQQ
NTPIGDGPVLLPDNHYLSYQSKLSKDPNEKRDHMVLLEFVTAAGITLGMDELYKSGLRSRAQ
ASNSAVDGTTSTRMLRVFILYAENVHTPDTDISDAYCSAVFAGVKKRTKVIKNSVNPVWNEG
FEWDLKGIPLDQGSELHVVVKDHETMGRNRFLGEAKVPLREVLATPSLSASFNAPLLDTKK
QPTGASLVLQVSYTPLPGAVLVKPFS

pV-2×C2Astop Plasmid

Using the primers shown below, Dysferlin C2A domain was added to pV-C2Astop by digestion ligation. The open reading frame includes venus (underlined)-C2A (italics).

### Primers Used for PCR:

DFkpnS (KpnI):
(SEQ ID NO: 1)
CGACggtaccactagtacgcgtATG.
C2AdoubKpnA (KpnI):
(SEQ ID NO: 5)
GCTAggtaccGCTGAAGGGCTTCACCAGCAC.
Nucleotide sequence:
SEQ ID NO: 6
ATGGTGAGCAAGGGCGAGGAGCTGTTCACCGGGGTGGTGCCCATCCTGGTCGAGCTG
GACGGCGACGTAAACGGCCACAAGTTCAGCGTGTCCGGCGAGGGCGAGGGCGATGCC
ACCTACGGCAAGCTGACCCTGAAGCTCATCTGCACCACCGGCAAGCTGCCCGTGCCCT
GGCCCACCCTCGTGACCACCCTCGGCTACGGCCTGCAGTGCTTCGCCCGCTACCCCG
ACCACATGAAGCAGCACGACTTCTTCAAGTCCGCCATGCCCGAAGGCTACGTCCAGGA
GCGCACCATCTTCTTCAAGGACGACGGCAACTACAAGACCCGCGCCGAGGTGAAGTTC
GAGGGCGACACCCTGGTGAACCGCATCGAGCTGAAGGGCATCGACTTCAAGGAGGAC
GGCAACATCCTGGGGCACAAGCTGGAGTACAACTACAACAGCCACAACGTCTATATCAC
CGCCGACAAGCAGAAGAACGGCATCAAGGCCAACTTCAAGATCCGCCACAACATCGAG
GACGGCGGCGTGCAGCTCGCCGACCACTACCAGCAGAACACCCCCATCGGCGACGGC
CCCGTGCTGCTGCCCGACAACCACTACCTGAGCTACCAGTCCAAGCTGAGCAAAGACC
CCAACGAGAAGCGCGATCACATGGTCCTGCTGGAGTTCGTGACCGCCGCCGGGATCAC
TCTCGGCATGGACGAGCTGTACAAGTCCGGACTCAGATCTCGAGCTCAAGCTTCGAATT
CTGCAGTCGACggtaccactagtacgcgtATGCTGAGGGTCTTCATCCTCTATGCCGAGAACGT
CCACACACCCGACACCGACATCAGCGATGCCTACTGCTCCGCGGTGTTTGCAGGGGTG
AAGAAGAGAACCAAAGTCATCAAGAACAGCGTGAACCCTGTATGGAATGAGGGATTTGA
ATGGGACCTCAAGGGCATCCCCCTGGACCAGGGCTCTGAGCTTCATGTGGTGGTCAAA
GACCATGAGACGATGGGGAGGAACAGGTTCCTGGGGGAAGCCAAGGTCCCACTCCGA
GAGGTCCTCGCCACCCCTAGTCTGTCCGCCAGCTTCAATGCCCCCCTGCTGGACACCA
AGAAGCAGCCCACAGGGGCCTCGCTGGTCCTGCAGGTGTCCTACACACCGCTGCCTG
GAGCTGTGCTGGTGAAGCCCTTCAGCggtaccactagtacgcgtATGCTGAGGGTCTTCATCCT
CTATGCCGAGAACGTCCACACACCCGACACCGACATCAGCGATGCCTACTGCTCCGCG
GTGTTTGCAGGGGTGAAGAAGAGAACCAAAGTCATCAAGAACAGCGTGAACCCTGTAT
GGAATGAGGGATTTGAATGGGACCTCAAGGGCATCCCCCTGGACCAGGGCTCTGAGCT
TCATGTGGTGGTCAAAGACCATGAGACGATGGGGAGGAACAGGTTCCTGGGGGAAGCC
AAGGTCCCACTCCGAGAGGTCCTCGCCACCCCTAGTCTGTCCGCCAGCTTCAATGCCC
CCCTGCTGGACACCAAGAAGCAGCCCACAGGGGCCTCGCTGGTCCTGCAGGTGTCCTA
CACACCGCTGCCTGGAGCTGTGCTGGTGAAGCCCTTCAGCTGA
Amino acid sequence:
SEQ ID NO: 7
MVSKGEELFTGVVPILVELDGDVNGHKFSVSGEGEGDATYGKLTLKLICTTGKLPVPWPTLV
TTLGYGLQCFARYPDHMKQHDFFKSAMPEGYVQERTIFFKDDGNYKTRAEVKFEGDTLVN
RIELKGIDFKEDGNILGHKLEYNYNSHNVYITADKQKNGIKANFKIRHNIEDGGVQLADHYQQ
NTPIGDGPVLLPDNHYLSYQSKLSKDPNEKRDHMVLLEFVTAAGITLGMDELYKSGLRSRAQ
ASNSAVDGTTSTRMLRVFILYAENVHTPDTDISDAYCSAVFAGVKKRTKVIKNSVNPVWNEG
FEWDLKGIPLDQGSELHVVVKDHETMGRNRFLGEAKVPLREVLATPSLSASFNAPLLDTKK
QPTGASLVLQVSYTPLPGAVLVKPFSGTTSTRMLRVFILYAENVHTPDTDISDAYCSAVFAG
VKKRTKVIKNSVNPVWNEGFEWDLKGIPLDQGSELHVVVKDHETMGRNRFLGEAKVPLREV
LATPSLSASFNAPLLDTKKQPTGASLVLQVSYTPLPGAVLVKPFS

pV-C2pkc-C2Astop Plasmid

Using the primers shown below, C2 domain of PKCα plus flanking sequences was added to pV-C2Astop by digestion ligation. The open reading frame includes venus (underlined)-C2pkc (underlined, italics)-C2A (italics).

### Primers Used for PCR:

PKCaC2AstopkpnS:
(SEQ ID NO: 8)
CGACggtaccactagtacgcgtATGGAGAAGAGGGGGGGGATTTAC.
PKCaC2AstopKpnA:
(SEQ ID NO: 9)
CGACggtaccGTTGCCAGCAGGGCCAAGTTTG.
Nucleotide sequence:
SEQ ID NO: 10
ATGGTGAGCAAGGGCGAGGAGCTGTTCACCGGGGTGGTGCCCATCCTGGTCGAGCTG
GACGGCGACGTAAACGGCCACAAGTTCAGCGTGTCCGGCGAGGGCGAGGGCGATGCC
ACCTACGGCAAGCTGACCCTGAAGCTCATCTGCACCACCGGCAAGCTGCCCGTGCCCT
GGCCCACCCTCGTGACCACCCTCGGCTACGGCCTGCAGTGCTTCGCCCGCTACCCCG
ACCACATGAAGCAGCACGACTTCTTCAAGTCCGCCATGCCCGAAGGCTACGTCCAGGA
GCGCACCATCTTCTTCAAGGACGACGGCAACTACAAGACCCGCGCCGAGGTGAAGTTC
GAGGGCGACACCCTGGTGAACCGCATCGAGCTGAAGGGCATCGACTTCAAGGAGGAC
GGCAACATCCTGGGGCACAAGCTGGAGTACAACTACAACAGCCACAACGTCTATATCAC
CGCCGACAAGCAGAAGAACGGCATCAAGGCCAACTTCAAGATCCGCCACAACATCGAG
GACGGCGGCGTGCAGCTCGCCGACCACTACCAGCAGAACACCCCCATCGGCGACGGC
CCCGTGCTGCTGCCCGACAACCACTACCTGAGCTACCAGTCCAAGCTGAGCAAAGACC
CCAACGAGAAGCGCGATCACATGGTCCTGCTGGAGTTCGTGACCGCCGCCGGGATCAC
TCTCGGCATGGACGAGCTGTACAAGTCCGGACTCAGATCTCGAGCTCAAGCTTCGAATT
CTGCAGTCGACggtaccactagtacgcgtATGGAGAAGAGGGGGCGGATTTACCTAAAGGCTGA
GGTTGCTGATGAAAAGCTCCATGTCACAGTACGAGATGCAAAAAATCTAATCCCTATGG
ATCCAAACGGGCTTTCAGATCCTTATGTGAAGCTGAAACTTATTCCTGATCCCAAGAATG
AAAGCAAGCAAAAAACCAAAACCATCCGCTCCACACTAAATCCGCAGTGGAATGAGTCC
TTTACATTCAAATTGAAACCTTCAGACAAAGACCGACGACTGTCTGTAGAAATCTGGGAC
TGGGATCGAACAACAAGGAATGACTTCATGGGATCCCTTTCCTTTGGAGTTTCGGAGCT
GATGAAGATGCCGGCCAGTGGATGGTACAAGTTGCTTAACCAAGAAGAAGGTGAGTAC
TACAACGTACCCATTCCGGAAGGGGACGAGGAAGGAAACATGGAACTCAGGCAGAAAT
TCGAGAAAGCCAAACTTGGCCCTGCTGGCAACggtaccactagtacgcgtATGCTGAGGGTCTT
CATCCTCTATGCCGAGAACGTCCACACACCCGACACCGACATCAGCGATGCCTACTGCT
CCGCGGTGTTTGCAGGGGTGAAGAAGAGAACCAAAGTCATCAAGAACAGCGTGAACCC
TGTATGGAATGAGGGATTTGAATGGGACCTCAAGGGCATCCCCCTGGACCAGGGCTCT
GAGCTTCATGTGGTGGTCAAAGACCATGAGACGATGGGGAGGAACAGGTTCCTGGGGG
AAGCCAAGGTCCCACTCCGAGAGGTCCTCGCCACCCCTAGTCTGTCCGCCAGCTTCAA
TGCCCCCCTGCTGGACACCAAGAAGCAGCCCACAGGGGCCTCGCTGGTCCTGCAGGT
GTCCTACACACCGCTGCCTGGAGCTGTGCTGGTGAAGCCCTTCAGCTGA
Amino acid sequence:
SEQ ID NO: 11
MVSKGEELFTGVVPILVELDGDVNGHKFSVSGEGEGDATYGKLTLKLICTTGKLPVPWPTLV
TTLGYGLQCFARYPDHMKQHDFFKSAMPEGYVQERTIFFKDDGNYKTRAEVKFEGDTLVN
RIELKGIDFKEDGNILGHKLEYNYNSHNVYITADKQKNGIKANFKIRHNIEDGGVQLADHYQQ
NTPIGDGPVLLPDNHYLSYQSKLSKDPNEKRDHMVLLEFVTAAGITLGMDELYKSGLRSRAQ
ASNSAVDGTTSTRMEKRGRIYLKAEVADEKLHVTVRDAKNLIPMDPNGLSDPYVKLKLIPDP
KNESKQKTKTIRSTLNPQWNESFTFKLKPSDKDRRLSVEIWDWDRTTRNDFMGSLSFGVSE
LMKMPASGWYKLLNQEEGEYYNVPIPEGDEEGNMELRQKFEKAKLGPAGNGTTSTRMLRV
FILYAENVHTPDTDISDAYCSAVFAGVKKRTKVIKNSVNPVWNEGFEWDLKGIPLDQGSELH
VVVKDHETMGRNRFLGEAKVPLREVLATPSLSASFNAPLLDTKKQPTGASLVLQVSYTPLP
GAVLVKPFS

pV-2×C2pkc-C2Astop Plasmid

Using the primers shown below, a second C2 domain of PKCα plus flanking sequences was added to pV-C2pkc-C2Astop by digestion ligation. The open reading frame includes venus (underline)-C2pkc (underline, italics)-C2A (italics).

### Primers Used for PCR:

PKCaC2AstopkpnS:
(SEQ ID NO: 8)
CGACggtaccactagtacgcgtATGGAGAAGAGGGGGGGGATTAC
PKCaC2AstopKpnA:
(SEQ ID NO: 9)
CGACggtaccGTTGCCAGCAGGGCCAAGTTTG.
Nucleotide sequence
(SEQ ID NO: 12)
ATGGTGAGCAAGGGCGAGGAGCTGTTCACCGGGGTGGTGCCCATCCTGGTCGAGCTG
GACGGCGACGTAAACGGCCACAAGTTCAGCGTGTCCGGCGAGGGCGAGGGCGATGCC
ACCTACGGCAAGCTGACCCTGAAGCTCATCTGCACCACCGGCAAGCTGCCCGTGCCCT
GGCCCACCCTCGTGACCACCCTCGGCTACGGCCTGCAGTGCTTCGCCCGCTACCCCG
ACCACATGAAGCAGCACGACTTCTTCAAGTCCGCCATGCCCGAAGGCTACGTCCAGGA
GCGCACCATCTTCTTCAAGGACGACGGCAACTACAAGACCCGCGCCGAGGTGAAGTTC
GAGGGCGACACCCTGGTGAACCGCATCGAGCTGAAGGGCATCGACTTCAAGGAGGAC
GGCAACATCCTGGGGCACAAGCTGGAGTACAACTACAACAGCCACAACGTCTATATCAC
CGCCGACAAGCAGAAGAACGGCATCAAGGCCAACTTCAAGATCCGCCACAACATCGAG
GACGGCGGCGTGCAGCTCGCCGACCACTACCAGCAGAACACCCCCATCGGCGACGGC
CCCGTGCTGCTGCCCGACAACCACTACCTGAGCTACCAGTCCAAGCTGAGCAAAGACC
CCAACGAGAAGCGCGATCACATGGTCCTGCTGGAGTTCGTGACCGCCGCCGGGATCAC
TCTCGGCATGGACGAGCTGTACAAGTCCGGACTCAGATCTCGAGCTCAAGCTTCGAATT
CTGCAGTCGACggtaccactagtacgcgtATGGAGAAGAGGGGGGGGATTTACCTAAAGGCTGA
GGTTGCTGATGAAAAGCTCCATGTCACAGTACGAGATGCAAAAAATCTAATCCCTATGG
ATCCAAACGGGCTTTCAGATCCTTATGTGAAGCTGAAACTTATTCCTGATCCCAAGAATG
AAAGCAAGCAAAAAACCAAAACCATCCGCTCCACACTAAATCCGCAGTGGAATGAGTCC
TTTACATTCAAATTGAAACCTTCAGACAAAGACCGACGACTGTCTGTAGAAATCTGGGAC
TGGGATCGAACAACAAGGAATGACTTCATGGGATCCCTTTCCTTTGGAGTTTCGGAGCT
GATGAAGATGCCGGCCAGTGGATGGTACAAGTTGCTTAACCAAGAAGAAGGTGAGTAC
TACAACGTACCCATTCCGGAAGGGGACGAGGAAGGAAACATGGAACTCAGGCAGAAAT
TCGAGAAAGCCAAACTTGGCCCTGCTGGCAACggtaccactagtacgcgtATGGAGAAGAGGG
GGCGGATTTACCTAAAGGCTGAGGTTGCTGATGAAAAGCTCCATGTCACAGTACGAGAT
GCAAAAAATCTAATCCCTATGGATCCAAACGGGCTTTCAGATCCTTATGTGAAGCTGAAA
CTTATTCCTGATCCCAAGAATGAAAGCAAGCAAAAAACCAAAACCATCCGCTCCACACTA
AATCCGCAGTGGAATGAGTCCTTTACATTCAAATTGAAACCTTCAGACAAAGACCGACG
ACTGTCTGTAGAAATCTGGGACTGGGATCGAACAACAAGGAATGACTTCATGGGATCCC
TTTCCTTTGGAGTTTCGGAGCTGATGAAGATGCCGGCCAGTGGATGGTACAAGTTGCTT
AACCAAGAAGAAGGTGAGTACTACAACGTACCCATTCCGGAAGGGGACGAGGAAGGAA
ACATGGAACTCAGGCAGAAATTCGAGAAAGCCAAACTTGGCCCTGCTGGCAACggtaccac
tagtacgcgtATGCTGAGGGTCTTCATCCTCTATGCCGAGAACGTCCACACACCCGACACCG
ACATCAGCGATGCCTACTGCTCCGCGGTGTTTGCAGGGGTGAAGAAGAGAACCAAAGT
CATCAAGAACAGCGTGAACCCTGTATGGAATGAGGGATTTGAATGGGACCTCAAGGGC
ATCCCCCTGGACCAGGGCTCTGAGCTTCATGTGGTGGTCAAAGACCATGAGACGATGG
GGAGGAACAGGTTCCTGGGGGAAGCCAAGGTCCCACTCCGAGAGGTCCTCGCCACCC
CTAGTCTGTCCGCCAGCTTCAATGCCCCCCTGCTGGACACCAAGAAGCAGCCCACAGG
GGCCTCGCTGGTCCTGCAGGTGTCCACCGCTGCCTGGAGCTGTGCTGGTGAAGCCCTT
CAGCTGA
Amino acid sequence:
SEQ ID NO: 13
MVSKGEELFTGVVPILVELDGDVNGHKFSVSGEGEGDATYGKLTLKLICTTGKLPVPWPTLV
TTLGYGLQCFARYPDHMKQHDFFKSAMPEGYVQERTIFFKDDGNYKTRAEVKFEGDTLVN
RIELKGIDFKEDGNILGHKLEYNYNSHNVYITADKQKNGIKANFKIRHNIEDGGVQLADHYQQ
NTPIGDGPVLLPDNHYLSYQSKLSKDPNEKRDHMVLLEFVTAAGITLGMDELYKSGLRSRAQ
ASNSAVDGTTSTRMEKRGRIYLKAEVADEKLHVTVRDAKNLIPMDPNGLSDPYVKLKLIPDP
KNESKQKTKTIRSTLNPQWNESFTFKLKPSDKDRRLSVEIWDWDRTTRNDFMGSLSFGVSE
LMKMPASGWYKLLNQEEGEYYNVPIPEGDEEGNMELRQKFEKAKLGPAGNGTTSTRMEK
RGRIYLKAEVADEKLHVTVRDAKNLIPMDPNGLSDPYVKLKLIPDPKNESKQKTKTIRSTLNP
QWNESFTFKLKPSDKDRRLSVEIWDWDRTTRNDFMGSLSFGVSELMKMPASGWYKLLNQ
EEGEYYNVPIPEGDEEGNMELRQKFEKAKLGPAGNGTTSTRMLRVFILYAENVHTPDTDISD
AYCSAVFAGVKKRTKVIKNSVNPVWNEGFEWDLKGIPLDQGSELHVVVKDHETMGRNRFL
GEAKVPLREVLATPSLSASFNAPLLDTKKQPTGASLVLQVSYTPLPGAVLVKPFS

pV-2×C2pkc-2×C2Astop Plasmid

Using the primers shown below, two consecutive C2 domains of PKCα plus flanking sequences were added to pV-2×C2Astop by digestion ligation. The open reading frame includes venus (underline)-C2pkc (underline, italics)-C2A (italics).

### Primers Used for PCR:

2xC2pkcsalS:
(SEQ ID NO: 14)
CGAGCTGTACAAGTCCGGACTCGTCCACAGATCTac.
2xC2pkcsa1A:
(SEQ ID NO: 15)
CGACTGCAGAATTCGAAGCTTTCAGTCGACGTTGCCAG:.
Nucleotide sequence:
SEQ ID NO: 16
ATGGTGAGCAAGGGCGAGGAGCTGTTCACCGGGGTGGTGCCCATCCTGGTCGAGCTG
GACGGCGACGTAAACGGCCACAAGTTCAGCGTGTCCGGCGAGGGCGAGGGCGATGCC
ACCTACGGCAAGCTGACCCTGAAGCTCATCTGCACCACCGGCAAGCTGCCCGTGCCCT
GGCCCACCCTCGTGACCACCCTCGGCTACGGCCTGCAGTGCTTCGCCCGCTACCCCG
ACCACATGAAGCAGCACGACTTCTTCAAGTCCGCCATGCCCGAAGGCTACGTCCAGGA
GCGCACCATCTTCTTCAAGGACGACGGCAACTACAAGACCCGCGCCGAGGTGAAGTTC
GAGGGCGACACCCTGGTGAACCGCATCGAGCTGAAGGGCATCGACTTCAAGGAGGAC
GGCAACATCCTGGGGCACAAGCTGGAGTACAACTACAACAGCCACAACGTCTATATCAC
CGCCGACAAGCAGAAGAACGGCATCAAGGCCAACTTCAAGATCCGCCACAACATCGAG
GACGGCGGCGTGCAGCTCGCCGACCACTACCAGCAGAACACCCCCATCGGCGACGGC
CCCGTGCTGCTGCCCGACAACCACTACCTGAGCTACCAGTCCAAGCTGAGCAAAGACC
CCAACGAGAAGCGCGATCACATGGTCCTGCTGGAGTTCGTGACCGCCGCCGGGATCAC
TCTCGGCATGGACGAGCTGTACAAGTCCGGACTCAGATCTCGAGCTCAAGCTTCGAATT
CTGCAGTCGACagatctactagtacgcgtATGGAGAAGAGGGGGGGGATTTACCTAAAGGCTGA
GGTTGCTGATGAAAAGCTCCATGTCACAGTACGAGATGCAAAAAATCTAATCCCTATGG
ATCCAAACGGGCTTTCAGATCCTTATGTGAAGCTGAAACTTATTCCTGATCCCAAGAATG
AAAGCAAGCAAAAAACCAAAACCATCCGCTCCACACTAAATCCGCAGTGGAATGAGTCC
TTTACATTCAAATTGAAACCTTCAGACAAAGACCGACGACTGTCTGTAGAAATCTGGGAC
TGGGATCGAACAACAAGGAATGACTTCATGGGATCCCTTTCCTTTGGAGTTTCGGAGCT
GATGAAGATGCCGGCCAGTGGATGGTACAAGTTGCTTAACCAAGAAGAAGGTGAGTAC
TACAACGTACCCATTCCGGAAGGGGACGAGGAAGGAAACATGGAACTCAGGCAGAAAT
TCGAGAAAGCCAAACTTGGCCCTGCTGGCAACAGATCTactagtacgcgtATGGAGAAGAGG
GGGCGGATTTACCTAAAGGCTGAGGTTGCTGATGAAAAGCTCCATGTCACAGTACGAGA
TGCAAAAAATCTAATCCCTATGGATCCAAACGGGCTTTCAGATCCTTATGTGAAGCTGAA
ACTTATTCCTGATCCCAAGAATGAAAGCAAGCAAAAAACCAAAACCATCCGCTCCACACT
AAATCCGCAGTGGAATGAGTCCTTTACATTCAAATTGAAACCTTCAGACAAAGACCGAC
GACTGTCTGTAGAAATCTGGGACTGGGATCGAACAACAAGGAATGACTTCATGGGATCC
CTTTCCTTTGGAGTTTCGGAGCTGATGAAGATGCCGGCCAGTGGATGGTACAAGTTGCT
TAACCAAGAAGAAGGTGAGTACTACAACGTACCCATTCCGGAAGGGGACGAGGAAGGA
AACATGGAACTCAGGCAGAAATTCGAGAAAGCCAAACTTGGCCCTGCTGGCAACGTCG
ACggtaccactagtacgcgtATGCTGAGGGTCTTCATCCTCTATGCCGAGAACGTCCACACACC
CGACACCGACATCAGCGATGCCTACTGCTCCGCGGTGTTTGCAGGGGTGAAGAAGAGA
ACCAAAGTCATCAAGAACAGCGTGAACCCTGTATGGAATGAGGGATTTGAATGGGACCT
CAAGGGCATCCCCCTGGACCAGGGCTCTGAGCTTCATGTGGTGGTCAAAGACCATGAG
ACGATGGGGAGGAACAGGTTCCTGGGGGAAGCCAAGGTCCCACTCCGAGAGGTCCTC
GCCACCCCTAGTCTGTCCGCCAGCTTCAATGCCCCCCTGCTGGACACCAAGAAGCAGC
CCACAGGGGCCTCGCTGGTCCTGCAGGTGTCCTACACACCGCTGCCTGGAGCTGTGCT
GGTGAAGCCCTTCAGCggtaccactagtacgcgtATGCTGAGGGTCTTCATCCTCTATGCCGAG
AACGTCCACACACCCGACACCGACATCAGCGATGCCTACTGCTCCGCGGTGTTTGCAG
GGGTGAAGAAGAGAACCAAAGTCATCAAGAACAGCGTGAACCCTGTATGGAATGAGGG
ATTTGAATGGGACCTCAAGGGCATCCCCCTGGACCAGGGCTCTGAGCTTCATGTGGTG
GTCAAAGACCATGAGACGATGGGGAGGAACAGGTTCCTGGGGGAAGCCAAGGTCCCA
CTCCGAGAGGTCCTCGCCACCCCTAGTCTGTCCGCCAGCTTCAATGCCCCCCTGCTGG
ACACCAAGAAGCAGCCCACAGGGGCCTCGCTGGTCCTGCAGGTGTCCTACACACCGCT
GCCTGGAGCTGTGCTGGTGAAGCCCTTCAGCTGA
Amino acid sequence:
SEQ ID NO: 17
MVSKGEELFTGVVPILVELDGDVNGHKFSVSGEGEGDATYGKLTLKLICTTGKLPVPWPTLV
TTLGYGLQCFARYPDHMKQHDFFKSAMPEGYVQERTIFFKDDGNYKTRAEVKFEGDTLVN
RIELKGIDFKEDGNILGHKLEYNYNSHNVYITADKQKNGIKANFKIRHNIEDGGVQLADHYQQ
NTPIGDGPVLLPDNHYLSYQSKLSKDPNEKRDHMVLLEFVTAAGITLGMDELYKSGLRSRAQ
ASNSAVDRSTSTRMEKRGRIYLKAEVADEKLHVTVRDAKNLIPMDPNGLSDPYVKLKLIPDP
KNESKQKTKTIRSTLNPQWNESFTFKLKPSDKDRRLSVEIWDWDRTTRNDFMGSLSFGVSE
LMKMPASGWYKLLNQEEGEYYNVPIPEGDEEGNMELRQKFEKAKLGPAGNRSTSTRMEK
RGRIYLKAEVADEKLHVTVRDAKNLIPMDPNGLSDPYVKLKLIPDPKNESKQKTKTIRSTLNP
QWNESFTFKLKPSDKDRRLSVEIWDWDRTTRNDFMGSLSFGVSELMKMPASGWYKLLNQ
EEGEYYNVPIPEGDEEGNMELRQKFEKAKLGPAGNVDGTTSTRMLRVFILYAENVHTPDTDI
SDAYCSAVFAGVKKRTKVIKNSVNPVWNEGFEWDLKGIPLDQGSELHVVVKDHETMGRNR
FLGEAKVPLREVLATPSLSASFNAPLLDTKKQPTGASLVLQVSYTPLPGAVLVKPFSGTTSTR
MLRVFILYAENVHTPDTDISDAYCSAVFAGVKKRTKVIKNSVNPVWNEGFEWDLKGIPLDQG
SELHVVVKDHETMGRNRFLGEAKVPLREVLATPSLSASFNAPLLDTKKQPTGASLVLQVSYT
PLPGAVLVKPFS

What is claimed:

1. A fusion protein engineered from a dysferlin C2 domain sequence linked to a sequence of a homologous fusion partner.

2. The fusion protein of claim 1, wherein the dysferlin C2 domain sequence is an N-terminal sequence or a C-terminal sequence.

3. The fusion protein of claim 2, wherein the dysferlin C2 domain sequence is an N-terminal C2A domain sequence (Dysf-C2A).

4. The fusion protein of claim 1, wherein the homologous fusion partner comprises a sequence from at least one C2 domain of an α isoform of protein kinase C (C2-PKCα).

5. The fusion protein of claim 1, wherein the fusion protein is an engineered C2-PKCα-DYSF-C2A fusion protein.

6. A vector construct comprising a cDNA encoding the fusion protein of claim 1.

7. A viral vector comprising the vector construct of claim 5 and a promoter effective to control expression of the fusion protein therein.

8. The viral vector of claim 6, wherein the promoter is a muscle-specific promoter.

9. A method for treating a dysferlinopathy in a subject in need thereof, comprising:
administering to the subject at least once a therapeutic amount of a viral vector that encodes a fusion protein comprising a dysferlin C2 domain sequence linked to a sequence of a homologous fusion partner to correct defects in a dysferlinopathic muscle, thereby treating the dysferlinopathy.

10. The method of claim 9, wherein the homologous fusion partner targets the dysferlin C2 domain sequence to triad junctions in a skeletal muscle.

11. The method of claim 9, wherein the fusion protein comprises the C2A domain of dysferlin and at least one C2 domain of an α isoform of protein kinase C (C2-PKCα-DYSF-C2A).

12. The method of claim 9, wherein the dysferlinopathy is muscular dystrophy.

13. A method for suppressing pathogenic Ca²⁺ signaling in a dysferlinopathic muscle, comprising:
delivering a fusion protein of a dysferlin C2 domain linked to a homologous fusion partner to target at least one triad junction in a dysferlinopathic muscle; and
activating the dysferlin C2 domain sequence upon targeting to the at least one triad junction to regulate Ca²⁺ signaling.

14. The method of claim 13, wherein the delivering step comprises contacting the dysferlinopathic muscle with a viral vector encoding the fusion protein.

15. The method of claim 13, wherein the fusion protein comprises the C2A domain of dysferlin and at least one C2 domain of an α isoform of protein kinase C (C2-PKCα-DYSF-C2A).

16. The method of claim 13, wherein the pathogenic Ca²⁺ signaling occurs in muscular dystrophy.

17. A method for suppressing pathogenic defects during membrane repair in a dysferlinopathic muscle, comprising:
contacting the dysferlinopathic muscle with a fusion protein of a dysferlin C2 domain linked to at least one homologous C2 domain.

18. The method of claim 17, wherein the contacting step comprises transfecting the dysferlinopathic muscle with a viral vector encoding the fusion protein to express the same.

19. The method of claim 17, wherein the fusion protein comprises the C2A domain of dysferlin and at least one C2 domain of an α isoform of protein kinase C (C2-PKCα-DYSF-C2A).

20. The method of claim 17, wherein the dysferlinopathic muscle is a muscle affected by muscular dystrophy.

21. A method for targeting proteins to triad junctions in skeletal muscles, comprising:
engineering a viral vector that encodes from a single cDNA encoding a fusion protein comprising a protein sequence of interest linked to a sequence homologous to the protein sequence that specifically targets the triad junctions;
delivering the viral vector to the skeletal muscles; and
encoding the fusion protein from the single cDNA, said fusion protein targeted to the triad junctions via the sequence homologous to the protein sequence.

22. The method of claim 21, wherein the encoding step is under the control of a muscle-specific promoter in the viral vector.

23. The method of claim 22, wherein the fusion protein comprises the C2A domain of dysferlin and at least one C2 domain of an α isoform of protein kinase C (C2-PKCα-DYSF-C2A).


SEQUENCE LISTING (17 sequences)

SEQ ID NO: 1 - DNA, 25 bp, Artificial Sequence
  Primer sequence for pV-C2Astop and pV-2xC2Astop plasmids
cgacggtaccactagtacgcgtatg

SEQ ID NO: 2 - DNA, 56 bp, Artificial Sequence
  Primer sequence for pV-C2Astop plasmid
atcagatatctcagctgaagggcttcaccagcacagctccaggcagcggtgtgtag

SEQ ID NO: 3 - DNA, 1191 bp, Artificial Sequence
  Nucleotide sequence for pV-C2Astop plasmid
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctcatctgcaccaccggcaagctgcccgtgccctggcccaccctcgtgaccaccctcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccactagtacgcgtatgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgctggtgaagcccttcagctga

SEQ ID NO: 4 - protein, 396 aa, Artificial Sequence
  Amino acid sequence for pV-C2Astop plasmid
MVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYKSGLRSRAQASNSAVDGTTSTRMLRVFILYAENVHTPDTDISDAYCSAVFAGVKKRTKVIKNSVNPVWNEGFEWDLKGIPLDQGSELHVVVKDHETMGRNRFLGEAKVPLREVLATPSLSASFNAPLLDTKKQPTGASLVLQVSYTPLPGAVLVKPFS

SEQ ID NO: 5 - DNA, 31 bp, Artificial Sequence
  Primer sequence for pV-2xC2Astop plasmid
gctaggtaccgctgaagggcttcaccagcac

SEQ ID NO: 6 - DNA, 1617 bp, Artificial Sequence
  Nucleotide sequence for pV-2xC2Astop plasmid
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctcatctgcaccaccggcaagctgcccgtgccctggcccaccctcgtgaccaccctcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccactagtacgcgtatgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgctggtgaagcccttcagcggtaccactagtacgcgtatgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgctggtgaagcccttcagctga

SEQ ID NO: 7 - protein, 538 aa, Artificial Sequence
  Amino acid sequence for pV-2xC2Astop plasmid
MVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYKSGLRSRAQASNSAVDGTTSTRMLRVFILYAENVHTPDTDISDAYCSAVFAGVKKRTKVIKNSVNPVWNEGFEWDLKGIPLDQGSELHVVVKDHETMGRNRFLGEAKVPLREVLATPSLSASFNAPLLDTKKQPTGASLVLQVSYTPLPGAVLVKPFSGTTSTRMLRVFILYAENVHTPDTDISDAYCSAVFAGVKKRTKVIKNSVNPVWNEGFEWDLKGIPLDQGSELHVVVKDHETMGRNRFLGEAKVPLREVLATPSLSASFNAPLLDTKKQPTGASLVLQVSYTPLPGAVLVKPFS

SEQ ID NO: 8 - DNA, 46 bp, Artificial Sequence
  Primer sequence for pV-C2pkc-C2Astop and pV-2xC2pkc-C2Astop plasmids
cgacggtaccactagtacgcgtatggagaagagggggcggatttac

SEQ ID NO: 9 - DNA, 32 bp, Artificial Sequence
  Primer sequence for pV-C2pkc-C2Astop and pV-2xC2pkc-C2Astop plasmids
cgacggtaccgttgccagcagggccaagtttg

SEQ ID NO: 10 - DNA, 1689 bp, Artificial Sequence
  Nucleotide sequence for pV-C2pkc-C2Astop plasmid
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctcatctgcaccaccggcaagctgcccgtgccctggcccaccctcgtgaccaccctcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccactagtacgcgtatggagaagagggggcggatttacctaaaggctgaggttgctgatgaaaagctccatgtcacagtacgagatgcaaaaaatctaatccctatggatccaaacgggctttcagatccttatgtgaagctgaaacttattcctgatcccaagaatgaaagcaagcaaaaaaccaaaaccatccgctccacactaaatccgcagtggaatgagtcctttacattcaaattgaaaccttcagacaaagaccgacgactgtctgtagaaatctgggactgggatcgaacaacaaggaatgacttcatgggatccctttcctttggagtttcggagctgatgaagatgccggccagtggatggtacaagttgcttaaccaagaagaaggtgagtactacaacgtacccattccggaaggggacgaggaaggaaacatggaactcaggcagaaattcgagaaagccaaacttggccctgctggcaacggtaccactagtacgcgtatgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgctggtgaagcccttcagctga

SEQ ID NO: 11 - protein, 562 aa, Artificial Sequence
  Amino acid sequence for pV-C2pkc-C2Astop plasmid
MVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYKSGLRSRAQASNSAVDGTTSTRMEKRGRIYLKAEVADEKLHVTVRDAKNLIPMDPNGLSDPYVKLKLIPDPKNESKQKTKTIRSTLNPQWNESFTFKLKPSDKDRRLSVEIWDWDRTTRNDFMGSLSFGVSELMKMPASGWYKLLNQEEGEYYNVPIPEGDEEGNMELRQKFEKAKLGPAGNGTTSTRMLRVFILYAENVHTPDTDISDAYCSAVFAGVKKRTKVIKNSVNPVWNEGFEWDLKGIPLDQGSELHVVVKDHETMGRNRFLGEAKVPLREVLATPSLSASFNAPLLDTKKQPTGASLVLQVSYTPLPGAVLVKPFS

SEQ ID NO: 12 - DNA, 2186 bp, Artificial Sequence
  Nucleotide sequence for pV-2xC2pkc-C2Astop plasmid
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctcatctgcaccaccggcaagctgcccgtgccctggcccaccctcgtgaccaccctcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccactagtacgcgtatggagaagagggggcggatttacctaaaggctgaggttgctgatgaaaagctccatgtcacagtacgagatgcaaaaaatctaatccctatggatccaaacgggctttcagatccttatgtgaagctgaaacttattcctgatcccaagaatgaaagcaagcaaaaaaccaaaaccatccgctccacactaaatccgcagtggaatgagtcctttacattcaaattgaaaccttcagacaaagaccgacgactgtctgtagaaatctgggactgggatcgaacaacaaggaatgacttcatgggatccctttcctttggagtttcggagctgatgaagatgccggccagtggatggtacaagttgcttaaccaagaagaaggtgagtactacaacgtacccattccggaaggggacgaggaaggaaacatggaactcaggcagaaattcgagaaagccaaacttggccctgctggcaacggtaccactagtacgcgtatggagaagagggggcggatttacctaaaggctgaggttgctgatgaaaagctccatgtcacagtacgagatgcaaaaaatctaatccctatggatccaaacgggctttcagatccttatgtgaagctgaaacttattcctgatcccaagaatgaaagcaagcaaaaaaccaaaaccatccgctccacactaaatccgcagtggaatgagtcctttacattcaaattgaaaccttcagacaaagaccgacgactgtctgtagaaatctgggatgggatcgaacaacaaggaatgacttcatgggatccctttcctttggagtttcggagctgatgaagatgccggccagtggatggtacaagttgcttaaccaagaagaaggtgagtactacaacgtacccattccggaaggggacgaggaaggaaacatggaactcaggcagaaattcgagaaagccaaacttggccctgctggcaacggtaccactagtacgcgtatgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgctggtgaagcccttcagctga

SEQ ID NO: 13 - protein, 728 aa, Artificial Sequence
  Amino acid sequence for pV-2xC2pkc-C2Astop plasmid
MVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYKSGLRSRAQASNSAVDGTTSTRMEKRGRIYLKAEVADEKLHVTVRDAKNLIPMDPNGLSDPYVKLKLIPDPKNESKQKTKTIRSTLNPQWNESFTFKLKPSDKDRRLSVEIWDWDRTTRNDFMGSLSFGVSELMKMPASGWYKLLNQEEGEYYNVPIPEGDEEGNMELRQKFEKAKLGPAGNGTTSTRMEKRGRIYLKAEVADEKLHVTVRDAKNLIPMDPNGLSDPYVKLKLIPDPKNESKQKTKTIRSTLNPQWNESFTFKLKPSDKDRRLSVEIWDWDRTTRNDFMGSLSFGVSELMKMPASGWYKLLNQEEGEYYNVPIPEGDEEGNMELRQKFEKAKLGPAGNGTTSTRMLRVFILYAENVHTPDTDISDAYCSAVFAGVKKRTKVIKNSVNPVWNEGFEWDLKGIPLDQGSELHVVVKDHETMGRNRFLGEAKVPLREVLATPSLSASFNAPLLDTKKQPTGASLVLQVSYTPLPGAVLVKPFS

SEQ ID NO: 14 - DNA, 36 bp, Artificial Sequence
  Primer sequence for pV-2xC2pkc-2xC2Astop plasmid
cgagctgtacaagtccggactcgtcgacagatctac

SEQ ID NO: 15 - DNA, 38 bp, artificial sequence
  primer sequence for pv-2xc2pkc-2xc2astop plasmid
cgactgcagaattcgaagctttcagtcgacgttgccag

SEQ ID NO: 16 - DNA, 2619 bp, Artificial Sequence
  Nucleotide sequence for pV-2xC2pkc-2xC2Astop plasmid
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctcatctgcaccaccggcaagctgcccgtgccctggcccaccctcgtgaccaccctcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacagatctactagtacgcgtatggagaagagggggcggatttacctaaaggctgaggttgctgatgaaaagctccatgtcacagtacgagatgcaaaaaatctaatccctatggatccaaacgggctttcagatccttatgtgaagctgaaacttattcctgatcccaagaatgaaagcaagcaaaaaaccaaaaccatccgctccacactaaatccgcagtggaatgagtcctttacattcaaattgaaaccttcagacaaagaccgacgactgtctgtagaaatctgggactgggatcgaacaacaaggaatgacttcatgggatccctttcctttggagtttcggagctgatgaagatgccggccagtggatggtacaagttgcttaaccaagaagaaggtgagtactacaacgtacccattccggaaggggacgaggaaggaaacatggaactcaggcagaaattcgagaaagccaaacttggccctgctggcaacagatctactagtacgcgtatggagaagagggggcggatttacctaaaggctgaggttgctgatgaaaagctccatgtcacagtacgagatgcaaaaaatctaatccctatggatccaaacgggctttcagatccttatgtgaagctgaaacttattcctgatcccaagaatgaaagcaagcaaaaaaccaaaaccatccgctccacactaaatccgcagtggaatgagtcctttacattcaaattgaaaccttcagacaaagaccgacgactgtctgtagaaatctgggactgggatcgaacaacaaggaatgacttcatgggatccctttcctttggagtttcggagctgatgaagatgccggccagtggatggtacaagttgcttaaccaagaagaaggtgagtactacaacgtacccattccggaaggggacgaggaaggaaacatggaactcaggcagaaattcgagaaagccaaacttggccctgctggcaacgtcgacggtaccactagtacgcgtatgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgctggtgaagcccttcagcggtaccactagtacgcgtatgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgctggtgaagcccttcagctga

SEQ ID NO: 17 - protein, 872 aa, Artificial Sequence
  Amino acid sequence for pV-2xC2pkc-2xC2Astop plasmid
MVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYKSGLRSRAQASNSAVDRSTSTRMEKRGRIYLKAEVADEKLHVTVRDAKNLIPMDPNGLSDPYVKLKLIPDPKNESKQKTKTIRSTLNPQWNESFTFKLKPSDKDRRLSVEIWDWDRTTRNDFMGSLSFGVSELMKMPASGWYKLLNQEEGEYYNVPIPEGDEEGNMELRQKFEKAKLGPAGNRSTSTRMEKRGRIYLKAEVADEKLHVTVRDAKNLIPMDPNGLSDPYVKLKLIPDPKNESKQKTKTIRSTLNPQWNESFTFKLKPSDKDRRLSVEIWDWDRTTRNDFMGSLSFGVSELMKMPASGWYKLLNQEEGEYYNVPIPEGDEEGNMELRQKFEKAKLGPAGNVDGTTSTRMLRVFILYAENVHTPDTDISDAYCSAVFAGVKKRTKVIKNSVNPVWNEGFEWDLKGIPLDQGSELHVVVKDHETMGRNRFLGEAKVPLREVLATPSLSASFNAPLLDTKKQPTGASLVLQVSYTPLPGAVLVKPFSGTTSTRMLRVFILYAENVHTPDTDISDAYCSAVFAGVKKRTKVIKNSVNPVWNEGFEWDLKGIPLDQGSELHVVVKDHETMGRNRFLGEAKVPLREVLATPSLSASFNAPLLDTKKQPTGASLVLQVSYTPLPGAVLVKPFS